(19) United States
(12) Patent Application Publication
(10) Pub. No.: US 2024/0384310 A1
(43) Pub. Date: Nov. 21, 2024

(54) PROCESS TO PRODUCE POLYHYDROXYALKANOATES FROM SEAWEED

(71) Applicant: C Sea Solutions Pty Ltd, Watermans Bay, Western Australia (AU)

(72) Inventors: Julia REISSER, Watermans Bay, Western Australia (AU); Michael KINGSBURY, Watermans Bay, Western Australia (AU); David CHUKA-OGWUDE, Watermans Bay, Western Australia (AU); Luke RICHARDS, Watermans Bay, Western Australia (AU); Aline Paternostro MARTINS, Watermans Bay, Western Australia (AU)

(73) Assignee: C Sea Solutions Pty Ltd, Watermans Bay, Western Australia (AU)

(21) Appl. No.: 18/577,315

(22) PCT Filed: Jul. 6, 2022
(86) PCT No.: PCT/AU2022/050706

§ 371 Date: Jan. 8, 2024

(30) Foreign Application Priority Data
Jul. 7, 2021 (AU) 2021902068
May 24, 2022 (AU) 2022901400

(51) Int. Cl. C12P 7/62

(52) U.S. Cl. CPC C12P 7/62

(57) ABSTRACT

A process is described for producing polyhydroxyalkanoates from macroalgae, the process comprising the steps of: forming a macroalgal mixture from macroalgae and a liquid; hydrolysing the macroalgae mixture to form a macroalgal hydrolysate; producing a growth medium comprising the macroalgal hydrolysate; fermenting the growth medium using halophilic microbes capable of producing polyhydroxyalkanoates; and extracting the polyhydroxyalkanoates from within the halophilic cells using a water-based, osmosis-driven lysis process.

### TECHNICAL FIELD

The present invention relates to processes of producing polyhydroxyalkanoates from seaweed by hydrolysis and fermentation using halophilic microbes.

### BACKGROUND ART

Polyhydroxyalkanoates (PHAs) are natural polyesters that can be derived from microbial fermentation of carbon from lignocellulosic and other biomass feedstocks. They are often referred to as ‘biopolymers’ and used to create ‘bioplastics’ due to their non-fossil fuel (petrochemical-based) sources.

In the microbial fermentation process, the microbes are often deprived of nutrients, for example, nitrogen, oxygen, and phosphorus, but provided with high levels of carbon. They produce PHAs as carbon reserves which they store in highly refractive granules for re-use when they have more of the other nutrients required to grow and reproduce. However, before the granules can be broken down, they can be harvested from the microbes by lysing the cells and the PHAs isolated. The yield of PHAs obtained from the intracellular granule inclusions can be as high as 80% of the organism's dry weight.

PHAs have a chemical structure similar to petrochemical-based plastics and because they are biodegradable, and will not harm living tissue, they have been used in agricultural, medical and pharmaceutical applications. For example, PHAs have been used to produce sutures, slings, bone plates and skin substitutes. PHAs have also been used for single-use food packaging. Bioplastics formed using PHAs are usually less toxic than petrochemical-based plastics and do not contain the hormone disrupter bisphenol A (BPA).

While production of PHAs has been investigated for more than 90 years, greater interest has arisen more recently in part due to greater public awareness of environmental issues from discarded plastics.

While bioplastics made from biopolymers including PHAs may seem to be one clear solution to the environmental devastation we are now experiencing from millions of tonnes of petrochemical-based plastics discarded in our waterways, in landfill, and from the microplastics in the food chain, they are not without their own pollution and sustainability issues.

Key sources of PHAs for producing bioplastics, amongst others, are currently from the fermentation of starch from corn, potatoes, cassava, and sugarcane, amongst other crops. These crops often utilise large tracts of arable land and if they are being used for biopolymer production, they are typically replacing food crops for our ever-growing populations. Thus, a first issue is the competition for finite land in general, but also for fertile arable land for food crops.

Further issues include the millions of litres of water needed to grow the crops, the fertilizers and pesticides which are synthetic and can pollute nearby waterways, and other environmental hazards of mass-production farming.

Chemical processing of the harvested crops for use in the production of PHAs also utilises large amounts of chemicals. Moreover, biopolymers still have significant greenhouse gas (GHG) footprints due: (i) to the need for carbon-intensive sterilisation of fermentation reactors including steam sterilisation which results in the release of high concentrations of CO2 equivalents and (ii) to the high carbon footprint associated with crop cultivation inputs (for example, fertilizers), amongst other things.

It would therefore be of benefit to provide more efficient and economical processes, while reducing the impact of these processes on the environment, for PHA production to become even more desirable as a replacement for petrochemical-based plastics.

The preceding discussion of the background art is intended to facilitate an understanding of the present invention only. It should be appreciated that the discussion is not an acknowledgement or admission that any of the material referred to was part of the common general knowledge as at the priority date of the application.

### SUMMARY OF THE INVENTION

The invention provides a process for producing polyhydroxyalkanoates (PHAs) from macroalgae, comprising the steps of:
forming a macroalgal mixture comprising macroalgae and a liquid;
hydrolysing the macroalgal mixture to form a macroalgal hydrolysate;
producing a growth medium comprising the macroalgal hydrolysate;
fermenting the growth medium using halophilic microbes capable of producing PHAs; and
extracting the PHAs from within the halophilic cells using a water-based, osmosis-driven lysis process.

For the purpose of describing the invention, halophilic microbes are those organisms that require salt in their growth media to survive, in contrast to ‘halotolerant’ microbes, which are organisms that can tolerate salt in their growth media.

The process of the invention as described herein uses halophilic species, which are not only advantageous due to their tolerance to salt present in seaweed, but also grow at such high levels of salt which inhibit most kinds of potential contamination during fermentation (thus decreasing product sterilization costs and associated carbon emissions). Using halophilic microbes also facilitates PHA product extraction from cells via osmotic shock lysis (using either freshwater or seawater).

In a preferred embodiment of the invention, the macroalgae comprises seaweed. More preferably the macroalgae comprises cultivated seaweed. Even more preferably the macroalgae comprises seaweed cultivated in industrial volumes. In this respect, of the approximately 25,000 seaweed species currently identified, only about 1% are currently cultivated and, of these, roughly 10 species are intensively used for large biomass production. Therefore, the process of the invention preferably utilises a seaweed from the four seaweed families that comprises approximately 95% of the current total world seaweed production: Gracilariaceae, Solieriaceae, Bangiaceae, and Laminariaceae.

In an embodiment of the invention, the macroalgae comprises a red macroalgae (Rhodophyta). More preferably, the macroalgae comprises a species of macroalgae selected from the group of families comprising: Gracilariaceae, Solieriaceae, Bangiaceae, Gelidiaceae, or Bonnemaisoniaceae. Even more preferably, the macroalgae comprises a species of macroalgae selected from the group comprising: Gracilaria spp., Gracilariopsis spp., Kappaphycus spp., Eucheuma spp., Porphyra spp., Pyropia spp., Gelidium spp., or Asparagopsis spp.

Alternatively, the macroalgae may comprise a species of green macroalgae (Chlorophyta), more preferably Ulva spp. The macroalgae may also comprise a brown macroalgae (Ochrophyta) selected from the family Laminariaceae or Lessoniaceae (kelps). More preferably, the species of brown macroalgae comprises Saccharina japonica (kombu), Undaria pinnatifida (wakame) or Ecklonia radiata (golden kelp).

In an embodiment of the invention, the macroalgal mixture comprises wet macroalgae and a liquid. For the purpose of describing the invention herein, ‘wet macroalgae’ refers to macroalgae that has not had its internal moisture content (completely) dried or substantially dried. That is, in some non-limiting examples, ‘wet macroalgae’ may comprise macroalgae that is fresh or has been freshly or recently cultivated, collected and/or removed from its growing environment, for example, a seawater or salt water environment; it may also comprise macroalgae that has been cultivated, collected and/or removed from its growing environment and then stored, frozen, cooled, and/or transported for a period of time prior to use in the process of the invention; it may also comprise wet macroalgae that has been spun down to remove excess water on the surface of the macroalgae during a cleaning process; and it may also comprise some degree of drying the macroalgae but not to completely remove the water content in the macroalgae.

In an embodiment, the wet macroalgae is collected and is not completely dried before the step of forming a macroalgal mixture. That is, in a preferred embodiment, the process of the invention utilises wet macroalgae collected or harvested from the water it had been living and growing in or even stored in, for example, oceans estuaries, bays, tanks, and ponds; with the seaweed grown and/or stored loose or attached to structures such as ropes. The macroalgae may be collected by hand or by mechanical or another means, for example, in a large-scale operation.

The wet macroalgae is preferably washed after collection with saline water or water from the site of collection which may comprise washing with water the macroalgae has been collected from.

The wet macroalgae is preferably cooled between collection and the step of forming a macroalgal mixture. Cooling preferably comprises storing the collected and washed macroalgae on ice or refrigerated container during storage and/or transport prior to the step in the process of the invention of forming a macroalgal mixture from wet macroalgae and a liquid.

Excess liquid is preferably drained off from the wet macroalgae before the step of forming a macroalgal mixture. More preferably, the excess liquid is drained off the wet macroalgae using a spinner.

Alternatively, in another embodiment of the process of the invention, the macroalgae may be dried. The macroalgae may be sun or solar dried, tunnel dried, freeze dried, vacuum or oven dried. Preferably, the macroalgae is solar or tunnel dried followed by freeze drying and/or oven-drying at between approximately 60 to 80° C., to remove remaining moisture.

In a preferred embodiment of the process of the invention, the step of forming a macroalgal mixture from (wet or dry) macroalgae and a liquid preferably comprises breaking down macroalgae into smaller portions in, and/or prior to mixing with, a liquid to form a macroalgal slurry of broken down macroalgae in the liquid. More preferably, breaking down macroalgae into smaller portions comprises the step of disintegrating macroalgae. Disintegrating macroalgae preferably comprises pulverizing by milling or blending macroalgae. Milling preferably comprises use of a knife or wet mill. When broken down into smaller portions, the smaller portions of macroalgae comprise particles of, preferably, less than approximately 2 mm in diameter. The macroalgae is, preferably, milled into a powder which enables it to be more easily hydrolysed.

In an embodiment of the process of the invention, lipids are extracted from the macroalgae, which has been broken down into smaller portions, prior to forming a mixture with the liquid.

Preferably, lipids are extracted by forming a mixture with the macroalgae and a solution that, preferably, contains chloroform, methanol, and water (including either deionised water, distilled water, reverse osmosis water, tap water, a saline solution and/or seawater). Preferably, that mixture contains 2 to 5% w/v macroalgae and, preferably, the chloroform-methanol-water solution contains between 35-45% chloroform, 35-45% methanol and 15-25% water.

The mixture is preferably mixed at room temperature for, preferably, between 10 mins to 40 mins and, more preferably, 30 mins, with the chloroform and methanolic phases separated from the macroalgae solids, preferably by centrifugation and/or filtration.

In an embodiment, the lipids content is extracted from the chloroform phase by evaporation and/or distillation, and the remaining macroalgae solids are, preferably, washed with deionised water, distilled water, reverse osmosis water, tap water, a saline solution and/or seawater, and the remaining solvents removed by centrifugation and/or filtration before the remaining macroalgae is combined with a liquid to form a macroalgal mixture.

In a preferred embodiment of the process of the invention, the liquid comprises a saline solution. For the purposes of describing the invention herein, ‘saline solution’ may comprise: salt water; water comprising salts whether artificially made, for example with distilled water and/or deionised water, or taken from a natural source; it may also comprise seawater whether unmodified, diluted in another liquid, for example, water, or concentrated to increase the concentration of salt or with addition of further salts; it may also comprise saline water from a salt water river or lake or other body of water; it may also comprise brackish, saline, or brine/briny water, for example, from desalination brine from a desalination process, for example, utilising reverse osmosis and/or evaporation processes; and it may also comprise a thalassic medium which may mimic or closely relate the composition of seawater.

In an embodiment of the invention, the saline solution comprises seawater. The seawater may comprise diluted or concentrated seawater.

In a preferred embodiment, the salinity of the saline solution is approximately 35 parts per thousand (ppt).

In an embodiment of the invention, the macroalgal mixture is stored at ambient temperature prior to hydrocolloid extraction as a preliminary step to prepare a fermentation growth medium. Preferably, the macroalgal mixture contains 1:3 to 1:50 w/v macroalgae and is stored for 30 mins to 24 hours. More preferably, the mixture contains 1:20 w/v macroalgae and is stored at ambient temperature for 3 hours.

In a preferred embodiment of the invention, the macroalgal mixture is heated and stirred and the aqueous phase containing hydrocolloids, for example, agar, is separated from the solids, preferably by filtration and/or centrifugation. Preferably, the mixture is heated to between 80° C. and 110° C. for, preferably, 1 to 4 hours and stirred at between 100 rpm to 1,100 rpm, prior to separation. More preferably, the mixture is heated to approximately 100° C. for approximately 2 hours and, preferably, stirred at 200 rpm, prior to separation.

In an embodiment, the solid residues remaining after the hydrocolloid extraction above are washed with a liquid, preferably at between 60° C. and 110° C., and, more preferably, approximately 70° C., to form another mixture and, preferably, the liquid comprises freshwater or a saline solution, to ensure any remaining hydrocolloids are extracted into the aqueous phase of the mixture and separated from the solid residues, preferably by filtration and/or centrifugation.

In an embodiment of the invention, the solid macroalgal residues are prepared for hydrolysis by mixing with liquid to form a further (second) macroalgal mixture. The separated liquid fraction, containing the hydrocolloid extracts, is allowed to gel, prior to a dehydration process to form a hydrocolloid product, and/or is hydrolysed to obtain additional sugar monomers such as galactose or glucose that may be used for fermentation processes such as to produce PHAs pursuant to the present invention.

In a preferred embodiment of the process of the invention, the step of hydrolysing the macroalgal mixture comprises one or more hydrothermal, acidic, and/or enzymatic hydrolysis processes. The hydrothermal process preferably comprises subcritical water extraction. The step of hydrolysing the macroalgal mixture preferably occurs either in batches or continuously, using one or a series of stirred reactor(s) and/or plug flow reactor(s).

In a preferred embodiment, the liquid is a saline solution. Preferably, the saline solution comprises a neat, concentrated or diluted solution, for example, seawater.

The hydrolysis of the macroalgal mixture is preferably within a hydrolysis reactor(s) or other suitable reactor(s).

In an embodiment, the step of acidic hydrolysis of the macroalgal mixture comprises a strong acid (for example, sulfuric acid, hydrochloric acid, and/or sulfamic acid) or a weak acid, preferably at a concentration of between approximately 0.1% to 5% w/v. The acid preferably comprises a weak acid and, more preferably, citric acid, acetic acid, formic acid, maleic acid, phosphoric acid, or oxalic acid. Even more preferably, the acid comprises citric acid. The concentration of citric acid comprises between approximately 10 to 200 mM, and more preferably approximately 25 mM.

In an embodiment, the hydrolysis of the macroalgal mixture, preferably comprising citric acidic hydrolysis, is conducted at between approximately 100° C. and 140° C., more preferably at approximately 120° C. The hydrolysis of the macroalgal mixture, preferably comprising citric acidic hydrolysis, is conducted for, preferably, approximately 10 min to 120 min, and more preferably for approximately 30 min and, preferably, stirred constantly at 100 rpm to 1,100 rpm, and more preferably at approximately 300 rpm. The aqueous phase is preferably separated from the macroalgal solids by centrifugation and/or filtration prior to enzymatic treatment.

In an embodiment, a detoxication process is performed on the aqueous phase to remove fermentation inhibitors prior to enzymatic treatment. This preferably includes over-liming treatment and/or charcoal treatment.

Over-liming preferably comprises adjusting the pH of the aqueous phase to pH 10 to 12 with a slurry of calcium hydroxide and water (including deionised water, distilled water, reverse osmosis water, tap water or saline solution), before separating the aqueous phase from the solids, preferably through centrifugation and/or filtration.

Charcoal treatment preferably consists of combining the aqueous phase with activated charcoal powder to form a mixture at a ratio of 0.5% to 10% w/v charcoal powder, and preferably 2.5% w/v, agitating the mixture (either by occasional or via constant stirring) for 10 to 120 mins, and preferably 30 mins, before separating the aqueous phase from the charcoal solids, preferably through centrifugation and/or filtration, and recombining with the acid-hydrolysed macroalgae solids to form a mixture.

In an embodiment, the step of enzymatic hydrolysis of the macroalgal mixture comprises adding enzymes to the acid-hydrolysed macroalgal mixture (including after detoxication if relevant). The enzymes preferably comprise one or more cellulases and/or a blend of beta-glucanases, pectinases, hemicellulases and xylanases. In a preferred embodiment, the enzymes comprise Celluclast® (‘Celluclast’) and Viscozyme® (‘Viscozyme’). The Celluclast and Viscozyme are preferably used at a rate of between approximately 0.01 to 2.0 ml enzyme/g macroalgae, more preferably approximately 0.1 ml of each of the two enzymes/g macroalgae for a total of approximately 0.2 ml enzyme/g macroalgae.

The enzymatic hydrolysis of the macroalgal mixture is preferably conducted at between approximately 30° C. and 70° C. at a pH of between approximately 3.5 and 7, and more preferably a pH of approximately 5.0. The enzymatic hydrolysis of the macroalgal mixture is more preferably conducted at approximately 50° C. The enzymatic hydrolysis of the macroalgal mixture is preferably conducted for between approximately 6 to 48 h, and more preferably 20 h, with mixing, wherein the mixing comprises stirring at approximately 200 rpm.

In an embodiment, the enzymatic hydrolysis of the macroalgal mixture produces an aqueous phase (‘macroalgal hydrolysate’) and a solid phase and the macroalgal hydrolysate is isolated from the solid phase. The step of isolating the macroalgal hydrolysate preferably comprises removal of the solid phase and residual solids by filtration or more preferably by centrifugation.

The post-hydrolysis solid phase is preferably further processed into a soil treatment or protein-rich food product suitable for human or animal consumption, and preferably as an aquaculture feed product.

In a preferred embodiment of the process of the invention, the macroalgal hydrolysate's pH is adjusted to approximately 7.0 by the addition of acid and/or base. The acid preferably comprises HCl and the base preferably comprises NaOH.

In a preferred embodiment of the process of the invention, the step of producing a growth medium for fermentation comprises adjusting the macroalgal hydrolysate's contents and properties to maximise growth and/or PHA production once inoculated with halophilic microbes. In a preferred embodiment, the halophilic microbe is a Haloferax spp., or more preferably H. mediterranei.

In an embodiment, the growth medium may be diluted with a saline solution to reduce fermentation inhibitor levels present in the macroalgal hydrolysate.

The salinity and nutrient content (including carbon, nitrogen and/or micronutrient levels) of the growth medium may also be adjusted through dilution, evaporation and/or via the addition of ingredients.

In an embodiment of the process, the salinity of the growth medium is adjusted to between 35 ppt and 330 ppt by the addition of a saline solution and/or salts (for example NaCl or sea salts). Preferably, where the halophilic microbe is H. mediterranei, the salinity of the growth medium is adjusted to between approximately 130 ppt and 200 ppt, and more preferably to approximately 170 ppt. The saline solution and/or salts preferably comprises sea salts and/or one or more of the following: NaCl, MgCl₂·6H₂O, Mg₂O₄·7H₂O, CaCl₂·6H₂O, NaHCO₃, and NaBr, and more preferably salts in proportions similar to ATCC Medium 1176 (Halobacterium medium).

In an embodiment of the process of the invention, the nutrient content and levels of the growth medium are adjusted by adding carbon sources (for example glucose, sucrose, and/or galactose), nitrogen sources (for example yeast extract, urea, ammonium chloride and/or ammonium sulfate), and/or micronutrients sources (for example trace elements mixes) into the growth medium. Preferably, where the halophilic microbe is H. mediterranei, the nutrient content of the growth medium is adjusted by adding yeast extract and/or SL-4 or SL-6 trace element mixes into the growth medium.

The step of fermenting the growth medium comprises inoculation of the growth medium with halophilic microbes capable of producing PHAs to form a fermentation culture. Inoculation volume is determined so that the inoculated fermentation culture has an optical density of between 0.5 to 1 (600 nm; OD₆₀₀).

In an embodiment, the growth medium is fermented in batches. In another embodiment, the growth medium is fermented in a continuous fermentation system.

In a preferred embodiment, the step of fermenting the growth medium comprises growing the fermentation culture in a continuous fermentation system using fermentation reactors.

In an embodiment, the continuous fermentation system comprises a fermentation reactor operating continuously or semi-continuously or two or more reactors operating sequentially, that is, in a continuous series or cascade.

The continuous fermentation system preferably comprises two or more reactors operating sequentially and, more preferably, with at least a first, a second, and a third fermentation reactor. The first fermentation reactor preferably contains a fermentation culture with composition optimised for cell growth. Downstream fermentation reactors (for example, the second and third fermentation reactors) preferably comprise fermentation cultures with compositions optimised to increase PHA accumulation within the cells.

Fermentation cultures inside the reactors are optimised by, preferably, increasing their carbon-nitrogen ratio from the first fermentation reactor to the last fermentation reactor. Each fermentation reactor also, preferably, has its carbon-nitrogen ratio kept at an optimal and constant level via continuous feeding of fresh growth medium into each reactor.

In an embodiment, where the halophilic microbe is H. mediterranei, the carbon-nitrogen ratio increases in the fermentation culture in each reactor from, preferably, a carbon-nitrogen ratio of between 1 to 20 in the first reactor to, preferably, a carbon-nitrogen ratio of between 21 to 40 in the last reactor.

The continuous fermentation system, preferably, comprises three inlet feeds continuously providing fresh growth media to the fermentation reactors (one per reactor) and three outlet feeds: one pumping culture from the first reactor to the second reactor, one from the second reactor to the third reactor, and one harvesting the fermentation broth from the third reactor for further processing into extracted PHAs. The last outlet feed is set at a flow rate equal to the sum of all inlet feed flow rates to maintain the fermentation culture volume within each reactor at a constant level.

In an embodiment, the third fermentation reactor feeds into a fourth reactor. The role of the fourth reactor is preferably to collect cell biomass for isolating the PHAs from the fermentation culture.

In an embodiment, acid and base are added during fermentation to maintain the pH at approximately pH 7.0. The acid preferably comprises HCl and the base preferably comprises NaOH. Antifoam is preferably added to the first, second, and third fermentation reactors to minimise production or accumulation of foam.

In an embodiment, the temperatures of the first, second, and third fermentation reactors are maintained at approximately 40° C. and preferably at atmospheric pressure.

In a preferred embodiment, the step of extracting the PHAs from within the halophilic cells using a water-based, osmosis-driven lysis process, starts with separating the cell biomass from the fermentation culture, preferably by centrifugation and/or filtration. Separating the cell biomass from the fermentation culture more preferably comprises centrifuging the fermentation culture at preferably 10,000 to 20,000 g.

The extracted halophilic cells are then lysed by inducing hypo-osmotic shock through submersion of the cells in a water-based solution with salinity lower than the cells' intracellular salinity. In an embodiment, the volume of the water-based solution used for each lysing stage is approximately the same volume as the volume of the separated fermentation culture (also known as the separated ‘broth’). Following lysis, PHAs, and any cells that have not yet lysed, are extracted, preferably, by filtration and/or centrifugation of the water-based solution containing the lysed, and any un-lysed, halophilic cells. For the purposes of describing the invention herein, this lysis and extraction process is defined as a ‘lysing stage’.

In an embodiment, a lysing stage is conducted one or more times and, preferably, once. The composition of the water-based solution that is used may be adjusted for each lysing stage depending on the number of lysing stages.

For the purposes of describing the invention herein, ‘water-based solution’ may comprise tap water, distilled water or deionised water that is either purchased or obtained on site, including via reverse osmosis or evaporation processes applied to seawater or freshwater sources. Alternatively, the water-based solution may comprise a saline solution. Preferably, for each lysing stage the water-based solution comprises 50 to 100% seawater, and more preferably, 99.9% seawater (or almost pure seawater) and 0.05 to 0.2% surfactant, where that surfactant is preferably sodium dodecyl sulfate (SDS).

In an embodiment, following the lysing stage extracted PHAs are then purified through submersion of the PHAs and any impurities (for example cell debris) in a further water-based solution. In an embodiment, the volume of the water-based solution used is, preferably, approximately the same volume as the volume of the water-based solution used for lysing. Following purification, PHAs are extracted, preferably, by filtration and/or centrifugation of the water-based solution containing the PHAs. For the purposes of describing the invention herein, this purification and extraction process is defined as a ‘purification stage’.

In an embodiment, a purification stage is conducted one or more times and, preferably, twice. The composition of the water-based solution that is used may be adjusted for each purification stage depending on the number of purification stages. Preferably, the water-based solution comprises 50 to 100% seawater, and more preferably, 100% seawater for each stage other than the last stage where it, preferably, comprises tap water, distilled water or deionised water that is either purchased or obtained on site, including via reverse osmosis or evaporation processes applied to seawater or freshwater sources.

PHAs that have been purified may be further treated to remove impurities and to be dried. The drying is preferably conducted between approximately 50° C. and 80° C., more preferably the drying is conducted at approximately 60° C. until a constant mass is reached, preferably vacuum dried.

The PHAs may be compounded with other ingredients, pelletized and/or extruded into fibres.

The herein described process of the invention is further distinct from other processes described in the art due to the following advantages, amongst others:

Firstly, the process of the invention uses cultivated or farmed macroalgae rather than terrestrial crops or waste streams as feedstock. By doing so, PHAs are derived from a crop that does not rely on unsustainable synthetic fertilizers nor scarce resources such as arable land or freshwater. Furthermore, since seaweed can be sustainably farmed over extensive ocean areas, it does not have the scalability issues often associated with the distributed nature of waste feedstocks such as dairy whey, waste lipids, sugar waste streams, and crop residues.

Given the climate and plastic pollution crises, there is an obvious need to decrease the world's dependency on fossil fuel-based chemicals and fuel commodities; and algae biorefineries are an attractive solution. In this respect, seaweed have potential to produce several billion tonnes of biomass per year and provide a sustainable supply of affordable and healthy products such as food, feed, fuel and biopolymers. Many seaweed are sugar-rich, contain none-to-minimal hard-to-process lignin, and grow quickly, at rates estimated to be at least three times faster than sugar cane and other land crops currently used for PHA production. Furthermore, some seaweed species are also already cultivated at scale for hydrocolloid production and human consumption. This is significant, not only because there's a ready supply of cultivated seaweed on the market to accelerate adoption, but also because the cultivation of those species is proven and scalable.

Secondly, since seaweed contain salts, the process of the invention employs, as an embodiment, saline hydrolysis, as well as halophilic microbes to produce biopolymers via thalassic fermentation. Because salt typically inhibits microbial growth, the process of the invention results in reduced contamination risks during the production process—from feedstock storage, through to hydrolysis and during fermentation. Accordingly, the process of the invention permits a continuous fermentation system for PHA production, with little downtime (that is both costly and slow) for (1) sterilisation and cleaning and (2) microbial lag phases in between fermentation batches. Importantly, the process also may use seawater, rather than freshwater, as a water source, increasing sustainability.

Thirdly, for many PHA producers today, the downstream extraction process is a key scale-up barrier because it typically requires large volumes of toxic chemicals and solvents (for example, chloroform) to extract PHA from cells. Those chemicals can (1) cost more than one third of the overall PHA production cost, (2) reduce PHA polymer purity, and (3) produce hazardous waste streams.

Importantly, therefore, the process of the invention, which uses a clean, water-based process to extract PHAs is valuable. Specifically, as a result of the presence of salt within halophilic cells (for example, in haloarchaea and halobacteria), lower salinity liquids (for example, freshwater and seawater) can be used to lyse cells via osmotic shock, releasing their contained PHA granules and minimising or eliminating the need for other chemicals or solvents.

Fourthly, the process of the invention generates valuable co-products without undermining PHA production yields. The first co-product, following hydrolysis, is a processed seaweed biomass with high protein content that can be used for human consumption and/or animal feed including, due to its salt content, aquaculture and fish feed. The second co-product is a hydrocolloid-rich extract that can be either sold to hydrocolloid off-takers, used to produce alternative biomaterials and/or break down into additional fermentable sugars. The third and final co-products are lipids extracted from macroalgae prior to hydrolysis. Those lipids often contain omega 3's and omega 6's and can be used for pharmaceutical and nutraceutical applications, amongst other things. Commercialising these co-products may generate significant economic and environmental returns, for example by the replacement of animal-based commodities with vegan ones.

It is worth highlighting that removing hydrocolloids from macroalgae upstream—for example, Ulvans from green macroalgae (Ochrophyta), Carrageenan and Agar from red macroalgae (Rhodophyta), and Alginates from brown macroalgae (Ochrophyta)—reduces the underlying chemical complexity of macroalgal biomass prior to hydrolysis. This leads to a more homogenous macroalgae hydrolysate. And, importantly, this means the process of the invention (1) is more scalable than other PHA processes because it can accommodate a wide range of feedstocks, including red, green and/or brown seaweed varieties, and (2) produces a PHA product that is more consist in terms of molecular weight and co-polymer composition (for example, in contrast to other PHA producers using variable organic waste streams, which generates variability in PHA product composition).

Finally, and importantly, the process of the invention generally removes/offsets more carbon pollution than is emitted during production. A key reason for this is because macroalgae sheds carbon-containing biomass into the water in which it grows, with a proportion of that detritus sequestered in sediments underneath farm sites and/or the deep ocean. Additionally, saline fermentation reduces sterilisation requirements, which reduces associated CO2 emissions compared that generated from intense sterilisation often required to support freshwater fermentation. As a result, the process of the invention produces not only carbon friendly PHAs that may substitute for carbon intensive plastics and bioplastics, but also co-products that may replace carbon intensive commodities such as meat-based foods and animal feeds. In aggregate, making the process of the invention ‘carbon negative’, thus, producing ‘carbon negative’ PHAs.

In this respect, the process of the invention comprises an effective tool for carbon sequestration and pollution mitigation. The seaweed can be cultivated using environmentally friendly methods (for instance, reference can be made to species cultivated at scale today such as Gracilaria spp, Kappaphycus spp. and Eucheuma spp.) Furthermore, in an embodiment, wet seaweed rather than dry seaweed may be used, which reduces energy and land requirements associated with industrial and sun drying respectively. Finally, commercial seaweed production often generates a number of major positive externalities, such as provisions of food, habitat and refuge for marine animals, ocean oxygenation, eutrophication remediation, reduction of harmful algal bloom events, reduction in greenhouse gases emissions from rearing herbivores, and jobs creation.

### BRIEF DESCRIPTION OF FIGURES

The present invention will now be described, by way of example, with reference to the accompanying drawings, in which:

FIG. 1. Report of chemical analysis of macroalgae hydrolysate.

FIG. 2. Table showing composition of salts in ATCC medium 1176 (Halobacterium medium).

FIG. 3. Table showing composition of (A) SL-6 and (B) SL-4 trace metal solutions added to the macroalgae hydrolysate-based growth medium.

FIG. 4. Bar graph showing sugar yield of fresh wet macroalgae hydrolysis (Gracilaria sp.) at varying salinity, temperature, and reaction time levels.

FIG. 5. Growth of H. mediterranei on various sugar substrates.

FIG. 6. Growth of H. mediterranei in dilutions of Gracilaria hydrolysate (33%, 25%, 17% strength) pre-treated with (A) citric acid, and (B) sulfuric acid.

FIG. 7. Growth of H. mediterranei in dilutions of Gracilaria hydrolysate (50%, 33%, 25%, 17% strength) with the addition of (A) no yeast extract, and (B) 2.5 g/L yeast extract.

FIG. 8. Changes in glucose concentration and absorbance (280 nm) during charcoal treatment of Gracilaria hydrolysate.

FIG. 9. Growth of H. mediterranei in dilutions of Gracilaria hydrolysate (50% and 33% strength) with and without charcoal treatment.

FIG. 10. Cell PHBV composition (% of dry weight) in reactors 1, 2 and 3 at a selected timepoint (121 h) during three-stage continuous culture.

FIG. 11. Photographs showing colour difference of agar extracted from seaweed when using freshwater (left) and seawater (right).

FIG. 12. Bar charts of 5-hydroxymethylfurfural (5-HMF) levels (in g/L of hydrolysate), as measured after (1) acid, (2) over-liming and (3) charcoal pre-treatments.

FIG. 13. Growth curves for H. mediterranei grown in 30 mL cultures in a shaker incubator (40° C., 150 rpm) with different growth media, (A) shows data for growth media made with non-diluted seaweed hydrolysate, and (B) shows data for growth media with diluted seaweed hydrolysate (50% strength). Different curves represent different levels of yeast extract added to the flask cultures, from 2 g/L (black line) to 1 g/L (darker gray), 0.3 g/L (medium gray) and 0 g/L (lighter curve).

FIG. 14. Bar chart of final PHBV readings (darker gray) and optical density readings (lighter gray) for H. mediterranei grown in different growth media. X-axis indicate whether growth media was prepared from diluted or undiluted seaweed hydrolysate, as well as concentration of yeast extract (g/L) in such growth media.

FIG. 15. Curves of growth and PHBV production (A), and nutrient consumption (B) time series of a semi-continuous fermentation using H. mediterranei in a 4 L bioreactor.

FIG. 16. Bar chart of biorefinery co-products with (darker gray) and without (lighter gray) the addition of a lipid extraction step. From right to left: grams of lipid extract, dry agar, glucose in hydrolysate, and protein in solids per 100 g of dry seaweed biomass.

FIG. 17. Grams of total protein, carbohydrate, and lipids/fat per 100 g of dry seaweed biomass. Darker gray bars show results for raw/initial seaweed biomass, while lighter gray bars indicate results for spent seaweed biomass/final solids.

FIG. 18. Growth curves of H. mediterranei in hydrolysate medium produced both with lipid extraction (dark) and without lipid extraction (light), monitored by optical density measurements.

FIG. 19. Photograph of pellet samples after first wash with solutions containing different seawater strengths. From left to right: 0% (100% RO water; sample 1), 12.5% (sample 2), 25% (sample 3), 37.5% (sample 4), 50% (sample 5), 62.5% (sample 6), 75% (sample 7), 87.5% (sample 8), and 100% (pure seawater-35 ppt; sample 9). The colour of the pellet ranges from a strong pink colour (sample 1; 100% freshwater) to very white (sample 9; 100% seawater).

### DESCRIPTION OF PREFERRED EMBODIMENTS

In order to provide a more precise understanding of the subject matter of the invention, features of the invention will now be discussed with reference to the following preferred embodiment or embodiments.

### First Preferred Embodiment

### Hydrothermal Hydrolysis and Batch Fermentation

Macroalgae Collection and Pre-Treatment. In a step of a first preferred embodiment of the process of the invention, seaweed (‘macroalgae’) of the species Gracilaria sp. that was growing within the estuary waters of the Swan River (Western Australia) was collected by hand.

The collected macroalgae biomass that was removed from the Swan River estuary was cleaned by washing the macroalgae on site using saline water from the Swan River to remove detritus from the surface of the macroalgae.

The cleaned macroalgae was then transported to the laboratory for processing. During transport, the temperature of the macroalgae was maintained at a temperature lower than ambient temperature within a cooler containing ice bricks.

At the laboratory, the macroalgae was manually drained using a 20 cm diameter salad spinner made of plastic. Seawater as a source of a saline solution was then added to the drained macroalgae and the macroalgae was then blended into small particles in a mechanical food blender (NINJA Blender with Auto-IQ BN495UK) via a 2 minute blend at the highest rpm available.

The amount of water that was added to the drained macroalgae was calculated based on the natural water content of the macroalgae (by measuring both the wet and dry weight of the macroalgae collected) as to result in a macroalgae mixture comprising a blended macroalgal slurry with an approximately 10% solid load composition. Using Gracilaria sp., approximately 200 ml of ‘artificial’ seawater was added per 500 g of fresh, wet (but drained) macroalgae biomass. The artificial seawater was produced using sea salts in reverse osmosis de-ionised water to achieve 35 ppt salinity.

The macroalgae mixture was refrigerated (approximately 4° C.) until further use.

Hydrolysis. In a following step of the process of the invention, the macroalgae mixture was hydrolysed using subcritical water hydrolysis (sub-CW). sub-CW is an ‘environmentally friendly’ method that utilises subcritical water to extract compounds of interest from the macroalgae. Subcritical water comprises water under high pressure and a temperature of between approximately 100° C. (boiling point for water) and 374° C. (the critical temperature of water).

The sub-CW hydrolysis was conducted in a 1 L reactor equipped with an electric heater, magnetic stirrer, analogue barometer, temperature probe, and controller.

The macroalgae mixture was hydrolysed using sub-CW in batches. For each batch, 700 g of macroalgae mixture was added into the reactor, some of the air was forced out of the reactor using an industrial vacuum pump (Sparmax TC-63 Dry Piston-Single Head Vacuum Pump) for 1 min, and the reactor was tightly capped.

A mechanical stirrer was cooled using a chiller utilising water at approximately 10° C. throughout the hydrolysis process to prevent the shaft bearing overheating. The air was evacuated from the reactor using the vacuum pump before every hydrolysis run on each batch.

The temperature of the reactor was set to 175° C., stirring to 280 rpm, and reaction time to 15 min.

After hydrolysis of each batch of macroalgae mixture, the hydrolysed macroalgae mixture was removed from the reactor and centrifuged at approximately 4000 g to separate the aqueous phase (‘macroalgae hydrolysate’) from the residual solids.

The macroalgae hydrolysate was filtered through a fiberglass filter (GFC; pore size 0.22 μm) using a suction filtration unit in preparation for use as a fermentation broth.

The residual solids were dried to a constant weight at approximately 60° C. for 48 h before being stored for future analysis and potential use as biochar or fertiliser.

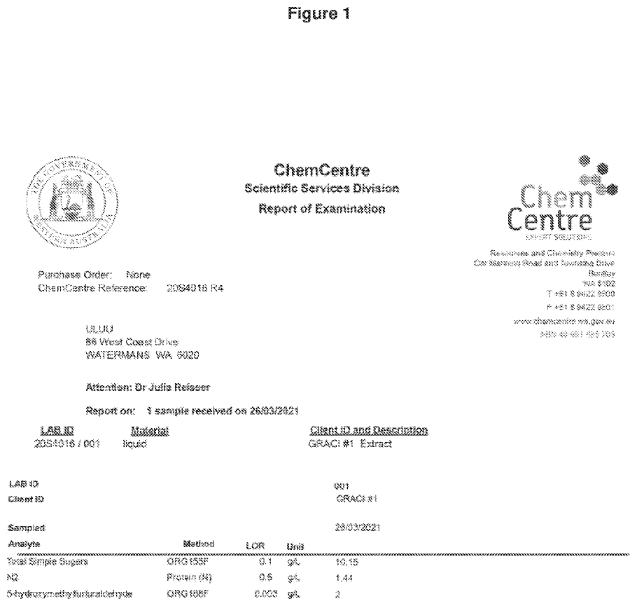

The macroalgae hydrolysate was then analysed (by The Government of Western Australia Chem Centre) which determined that the macroalgae hydrolysate contained 10.15 g/L of fermentable sugars, 1.44 g/L of protein nitrogen and 2 g/L of 5-hydroxymethylfurfuraldehyde (5-HMF). The results are shown in the report provided by the Chem Centre in FIG. 1.

Further analysis by the University of Western Australia determined that the monosaccharides in the hydrolysate comprised 10.3% xylose, 12.7% mannose, 51.6% galactose, and 25.4% glucose.

Microorganism for Fermentation. The microorganism used in the fermentation step of the process of the invention to produce macroalgae-originating PHAs were the haloarchaea Haloferax mediterranei from the American Type Culture Collection (ATCC 33500).

H. mediterranei is capable of producing PHAs from a wide variety of different carbon feedstock types, and it also has the ability to naturally produce PHBVs (poly(3-hydroxybutyrate-co-3-hydroxyvalerate)), which are PHA-type polymers with enhanced mechanical and physical properties. For instance, PHBV is typically an order of magnitude more elastic than PHB (polyhydroxybutyrate).

H. mediterranei thrives in very high salinity environments and has been shown to accumulate more polymer than other halophilic microorganisms.

The salinity in the growth medium for H. mediterranei is sufficiently high that very few other microorganisms, if any that may be present in the vicinity, can develop at growth rates anywhere near as high as those reached by H. mediterranei to grow to any significant numbers.

Therefore, the requirement for sterile conditions when using H. mediterranei is greatly reduced, and extremely simple production systems may be developed, including as one example, open ponds similar to those used in sewage treatment.

Fermentation. In a following step of the process of the invention, fermentation of the macroalgae hydrolysate was conducted in batches in a 6 L Minifors 2 bioreactor (4 L working volume).

Macroalgae hydrolysate-based growth medium for a batch was prepared by mixing 1 L of macroalgae hydrolysate with 1.25 g/L of yeast extract (as to increase nitrogen and other nutrient levels in the medium) and 3 L of water (as to reduce the concentration of fermentation inhibitors, for example, 5-HMF) to form a fermentation culture.

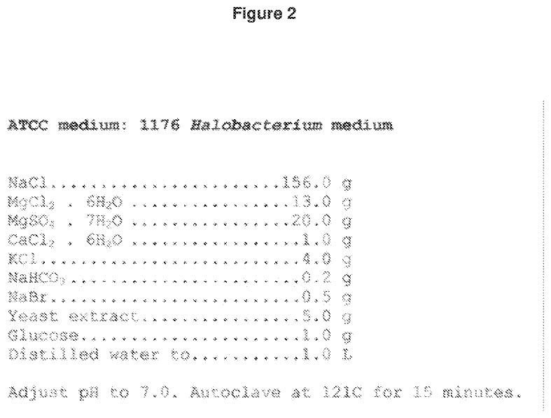

The resulting fermentation culture had its pH adjusted to approximately 7.0. Salinity of the fermentation culture was increased to approximately 170 ppt salinity via addition of salts at proportions equivalent to ATCC Medium 1176 (Halobacterium medium) and according to FIG. 2.

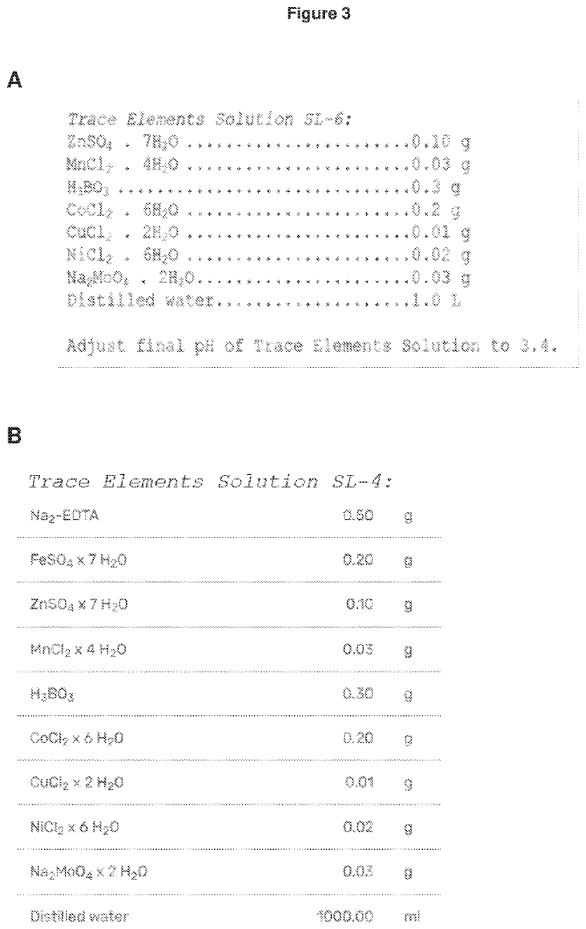

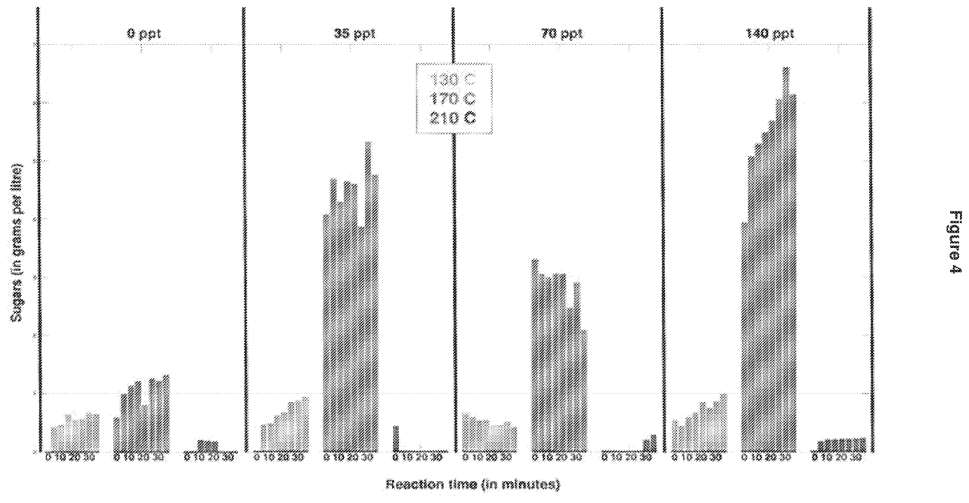

SL-6 solution of the composition as shown in FIG. 3 has been shown to improve uptake of galactose by H. mediterranei when using cheese whey as carbon source. To take benefit from this finding, 0.1% of an SL-6 solution was added to the fermentation culture.

The temperature of the bioreactor was set at 40° C. The pH of the macroalgae fermentation culture was maintained at approximately 7.0 by the automatically controlled addition of base in the form of 5% (w/v) NaOH and acid in the form of 5% (w/v) H₂SO₄.

Dissolved oxygen in the fermentation culture was maintained at approximately 20% by automatically controlled variation of the stirring speed, between approximately 200 rpm and 650 rpm, and aeration of up to approximately 6 L/min.

Foaming of the stirred fermentation culture in the bioreactor was suppressed by the automatically controlled addition of Antifoam A (Sigma®).

The fermentation of a batch in the bioreactor was initiated by addition of an approximately 2.5% v/v inoculum of H. mediterranei to the fermentation culture which equated to approximately 100 ml at 50 g/L culture density.

A fermentation batch of the fermentation culture was carried out in the bioreactor for a period of 96 h.

Polymer Extraction and Identification. After the period of fermentation has been completed, the next step of polymer extraction was conducted on a fermentation batch by hypo-osmotic shock.

The fermentation culture was centrifuged for 15 min at 4307 g.

The supernatant was discarded, and the remaining cell pellet washed with a 10% w/v NaCl solution.

The cell pellet was then washed multiple times with deionized water (with a proportion of 1 g cell wet mass per 20 ml water) causing hypo-osmotic shock until a cell pellet of a ‘whitish’ colour was obtained. In each wash, the cell debris from cells lysed during the hypo-osmotic shock that floated to the surface of the resulting supernatant was discarded. The deionised water was obtained via reverse osmosis.

The ‘whitish’ pellet of PHAs remaining at the completion of the washing procedure was then dried at 70° C. until a constant mass was achieved.

Fourier-transform infrared (FT-IR) spectroscopy of PHAs in the dried cell pellet was measured on a spectrometer equipped with Attenuated Total Reflectance attachment in a spectral range of 400 to 4000 cm-1. The measurement confirmed that the extracted PHAs comprise PHBVs.

### Adjusting Hydrolysis Variables for Improved Sugar Recovery

An important step in seaweed biomass conversion to PHAs is the depolymerization of the polysaccharides to produce monosaccharides. These sugars are the carbon sources for PHA production via fermentation.

Current experimentation is to extract sugars from the seaweed biomass using subcritical-water (sub-CW) hydrolysis which does not involve the use of any hazardous and/or costly chemicals, and it occurs in water heated to relatively mild temperatures (100° C.-240° C.).

In the above described first preferred embodiment of the process of the invention described above, the parameters previously used comprised:
Reaction temperature: 170° C.;
Solid load: 10% w/v (obtained by mixing 500 g of fresh wet Gracilaria spp. with 200 ml of artificial seawater);
Macroalgal mixture water salinity: 35 ppt;
Reaction time: 15 min (plus heat-up and cool-down times). Heat-up and cool-down were relatively long, with approximately 70 min and 30 min, respectively (for subsequent experimentation these heat-up and cool-down time periods should be made as short as possible as they can potentially compromise the quality of the resulting seaweed hydrolysate);
Hydrolysate Sugar and Nitrogen contents-Chem Centre analysis identified the seaweed hydrolysate had a protein nitrogen content equal to 0.14%; and Chem Centre and UWA HPLC analysis identified that the seaweed hydrolysate had approximately 10 g/L of total sugars.

In further experimentation, different parameters were varied to improve the resulting sugar recovery from Gracilaria sp. via sub-CW hydrolysis.

Parameters not varied comprised seaweed species being Gracilaria sp. from the Swan River, and a macroalgal mixture with a solid load of approximately 10% w/v.

Parameters varied comprised:
Reaction temperature of 130° C., 170° C., 210° C.;
Reaction time=0, 5, 10, 15, 20, 25, 30, 35 min at target temperature; and
Macroalgae mixture water salinity=0, 35, 70, 140 ppt. These salinities have relevant operational implications for the process to determine the water composition that functions more preferably as a catalyst to break-down seaweed carbohydrates into fermentable sugars.

Seaweed Collection. Gracilaria spp. specimens were collected at Point Resolution (Swan River estuary) by hand and placed inside a cooler with local water and ice blocks for transportation. Once in the lab, the seaweed biomass was drained with a salad spinner, placed inside resealable plastic bags (around 200 g per bag), and stored at −20° C. until use.

The experiment involved 12 batch reactions, each using 500 g of fresh Gracilaria sp. (see details under ‘Hydrolysis’ below). Therefore, 6 kg of Gracilaria sp. biomass (wet weight) was collected.

Macroalgae Mixture Preparation. To prepare samples for hydrolysis, the wet seaweed biomass was defrosted and separated into four 1.5 kg samples. Each of these samples was mixed with water to form a macroalgal mixture, which was blended to form a macroalgal slurry by finely grinding the mixture using a mechanical blender.

The water was prepared to be added to the seaweed biomass by using reverse osmosis deionised (RODI) water and Red Sea salt. Four different solutions comprising different salinities were made up for adding to the seaweed biomass to make four macroalgal slurries:

Solution 1: 0 ppt
Solution 2: 35 ppt
Solution 3: 70 ppt
Solution 4: 140 ppt

The amount of each solution added to the seaweed was calculated based on the natural seaweed water content (calculated by measuring both the wet and dry weight of the seaweed collected) as to result in a slurry with a ˜10% (w/v) solid load composition. In the case of Gracilaria sp. approximately 200 ml of water was added per 500 g of wet seaweed biomass. These seaweed slurries were stored at −20° C. until further use.

Hydrolysis. The sub-CW hydrolysis was conducted in a 1 L reactor equipped with an electric heater, mechanical stirrer, analogue pressure gauge, temperature probe and controller. Stirring was set to 70 rpm.

For each batch, 700 g of seaweed slurry was added into a 1 L reactor which was capped tightly, and then some of the air was forced out of the reactor using an air pump. The bearing of the mechanical stirrer was cooled using a chiller (10° C. water) throughout the hydrolysis process.

There were 12 batch hydrolysis runs, each with a unique combination of temperature, salinity and reaction time. For each batch, 10 samples were collected throughout the reaction as to identify optimal residence time. The first sample was the raw seaweed slurry (used to calculate sugar yields), sample two was collected when the reaction temperature reached 50% of the target temperature, and samples 3 to 10 were respectively collected at reaction times=0, 5, 10, 15, 20, 25, 30, and 35 min.

Analysis. Samples were filtered through a 0.2 μm filter, with the macroalgae hydrolysate stored at −20° C. and the solids dried at 60° C. for 48 h and then stored at ambient temperatures.

The amount of total soluble sugars in the macroalgae hydrolysate was analysed by the University of Western Australia and shown in the bar graph of FIG. 4.

Optimal temperature for producing highest sugars content following hydrolysis was shown to be approximately 170° C. when compared to results at 130° C. or 210° C.

Optimal salinity for producing highest sugars content following hydrolysis was shown to be approximately 140 ppt when compared to 0 ppt, 35 ppt and 70 ppt.

### Second Preferred Embodiment

### Enzymatic Hydrolysis and Continuous Fermentation

Macroalgae Sourced. In a step of a second preferred embodiment of the process of the invention, ‘farmed’ macroalgae (seaweed) of the species Gracilaria sp. that was growing in the waters off islands of Indonesia was collected by hand. The water on the collected macroalgae was removed by sun drying to reduce weight for transport and the macroalgae was transported by shipping container to Perth, Australia in vacuum-sealed bags.

Pre-treatment. Once received at the processing facility in Perth, Australia, the macroalgae was stored until processing in dry, dark and cold storage.

The macroalgae was freeze-dried in a lyophilizer (Model: BK-FD10S, Biobase Biodustry (Shandong) Co. Ltd) comprising a cooling trap that operates at −65° C. Approximately 15% of the original weight was lost in this step due to the removal of water from the seaweed.

Before proceeding to the hydrolysis stage, it was necessary to reduce the size of the raw macroalgae material as it arrived in large pieces. The macroalgae was first chopped in a shredder (Model: RSH2445S, Ryobi) to achieve a macroalgae particle size of between approximately 5 cm or less.

Then a second size reduction was performed in a food processor (Model: Blixer 7 v.v., Robot Coupe) to reduce the size of the macroalgae to particles of equal to or less than approximately 1 mm, i.e. into a powdered macroalgae form.

Hydrolysis. The hydrolysis of the macroalgae combined acid and enzymatic methods.

A batch of hydrolysate was prepared in a hydrolysis reactor (Model: FCF-10L, Zhengzhou Keda Machinery and Instrument Equipment Co. Ltd) with 7 litres of freshwater and 700 grams of seaweed powder (i.e. a 10% w/v loading).

A solution of citric acid (25 mM, ˜4.8 g of citric acid powder/L of solution) was added to the hydrolysis reactor.

The mixture was then pre-heated with mixing for 2 h at 120° C.

After the pre-heating, the temperature in the hydrolysis reactor was reduced to 55° C. by circulating glycol through the inner coil of the hydrolysis reactor. Once the temperature was adjusted to 55° C., the pH was adjusted to 5 by adding approximately 14 ml of a solution of NaOH (2 M) per litre of solution.

A 1:1 blend of Viscozyme and Celluclast enzyme mixtures (approximate density 1.1 g/ml) was added to the hydrolysate (approximately 0.2 ml per gram of seaweed) and the reaction left for 24 h with stirring at a velocity of 200 rpm.

Upon completion of the 24 h reaction time, the next step was to remove the solids from the hydrolysis reaction mixture. This was carried out by centrifuging (Model: 5910-R, Eppendorf) the hydrolysis reaction mixture for 30 min at 15,000 g. The macroalgae hydrolysate was then decanted from the centrifuged hydrolysis reaction mixture.

Certain fermentation inhibitors are typically generated during the hydrolysis of macroalgae, for example, when sugars are degraded, and other inhibitors are already present, for example, for use in plant defence. These inhibitors can include 5-HMF, organic acids, and polyphenolic compounds such as flavonoids. To remove such inhibitors, the macroalgae hydrolysate was treated with charcoal for 60 min in the hydrolysis reactor with 175 g of granulated charcoal (2.5% w/v) added to the macroalgae hydrolysate. Then, the used charcoal was removed through centrifugation for 30 min at 15,000 g.

Completion of the hydrolysis stage comprised the addition of salts to produce a suitable fermentation medium. Salts were added to the hydrolysate to give the composition provided for in ATCC Medium 1176 (as shown in FIG. 2, without yeast extract or additional glucose) and then 1 ml per litre of SL-4 trace metals mix (FIG. 3) was added. The salts were dissolved by mixing at 200 rpm in the hydrolysis reactor.

The final volume of the macroalgae hydrolysate after the hydrolysis was 7.5 L with a concentration of 20 g/L glucose.

Fermentation. The fermentation system is a continuous fermentation system comprising four fermentation bioreactors (5 L capacity):
Bioreactor 1: Minifors 2, Infors HT
Bioreactor 2: 5 L glass fermenter, Shanghai Bailun Biotechnology Co. Ltd.
Bioreactor 3: 5 L glass fermenter, Shanghai Bailun Biotechnology Co. Ltd.
Bioreactor 4: 5 L glass fermenter, Shanghai Bailun Biotechnology Co. Ltd.

The four fermentation bioreactors had independent feed solutions. The feed solutions consisted of dilutions of the macroalgae hydrolysate mixed with salts as described above. The hydrolysates were diluted with a solution containing salts addition at proportions equivalent to ATCC Medium 1176 (FIG. 2; without yeast extract or glucose) with the addition of 1 mL per litre of SL-4 trace metals mix described in FIG. 3) according to Table 1. The purpose of the fourth bioreactor was to collect cells for harvest and extraction of PHAs.

TABLE 1
Composition of the bioreactor feeds.

| Feed | Hydrolysate strength (%) |
|---|---|
| Bioreactor 1 | 50% |
| Bioreactor 2 | 75% |
| Bioreactor 3 | 100% |
| Bioreactor 4 | No feed |

The bioreactors were initially filled with 3.5 L of fermentation medium consisting of 50% strength hydrolysate. The bioreactors were each inoculated with an approximately 6% v/v inoculum of H. mediterranei and ran in batch mode until exhaustion of glucose. The bioreactors were then connected and ran in continuous mode with feed flow rates and operating conditions as described in Table 2.

TABLE 2
Feed flow rates and operating conditions of the four bioreactors.

| | Bioreactor 1 | Bioreactor 2 | Bioreactor 3 | Bioreactor 4 |
|---|---|---|---|---|
| Feed flow | 0.087 | 0.022 | 0.022 | — |
| rate (L/h) | | | | |
| Dilution rate | 0.025 | 0.031 | 0.037 | — |
| (h⁻¹) | | | | |
| Working | 3.5 | 3.5 | 3.5 | 0-4 |
| volume (L) | | | | |
| Temperature | 40 | 40 | 40 | 40 |
| (° C.) | | | | |
| pH | 7 | 7 | 7 | 7 |
| Dissolved | 40 | 40 | 40 | 40 |
| oxygen (%) | | | | |

Harvesting. Fermentation broth was periodically removed from Bioreactor 4. The biomass was then separated from the broth by centrifugation (Model: 5910-R, Eppendorf) at 15000 g for 15 min. Around 1% of the solution was recovered in biomass.

After the centrifugation, the biomass was treated in a cytolysis reactor (Model: 5 L glass fermenter, Shanghai Bailun Biotechnology Co. Ltd) in which a solution of 0.1% sodium dodecyl sulphate (SDS) was added to the reactor. The amount of SDS solution added was equivalent to the amount of broth removed from the centrifugation (i.e. 3.5 L). The cytolysis was performed for 1 h at 1100 rpm with a magnetic stirrer.

The solution was centrifuged (Model: 5910-R, Eppendorf) at 15000 g for 15 min to recover the PHAs.

To remove any residual SDS, a washing step was performed by adding 3.5 L of freshwater to the PHA pellet after the broth was removed and stirring the solution for 1 h at 1100 rpm with a magnetic stirrer. The solution was then centrifuged again to recover the PHAs in a centrifuge (Model: 5910-R, Eppendorf) at 15000 g for 15 min.

Around 30% of the recovered biomass corresponded to the collected PHAs.

The harvesting stage was completed by removing the remaining moisture in the PHA pellet in a vacuum oven at 60° C.

### Determining Preferred Conditions for Steps of Process According to Second Preferred Embodiment

Growth of Haloferax mediterranei on different carbon sources. As the sugar subunits composing macroalgae polysaccharides vary between red, green and brown seaweeds, experiments were conducted to identify how different sugars performed as substrates for H. mediterranei growth to inform seaweed selection for PHA production.

Nine different monosaccharides found in macroalgal polysaccharides (glucose, rhamnose, arabinose, galactose, xylose, glucuronic acid, mannitol, fucose, mannose) were selected. Growth media was prepared by the addition of 10 g/L of each sugar to a basal medium comprised of ATCC Medium 1176 (FIG. 2; prepared without glucose). A volume of 60 mL of each medium was transferred to a 250 mL flask and inoculated with H. mediterranei cells (grown overnight in ATCC Medium 1176) to an optical density (600 nm; OD₆₀₀) of approximately 0.5. Flasks were incubated in a shaker incubator at 40° C. and 150 rpm and monitored by OD₆₀₀ measurements over a 180 h period.

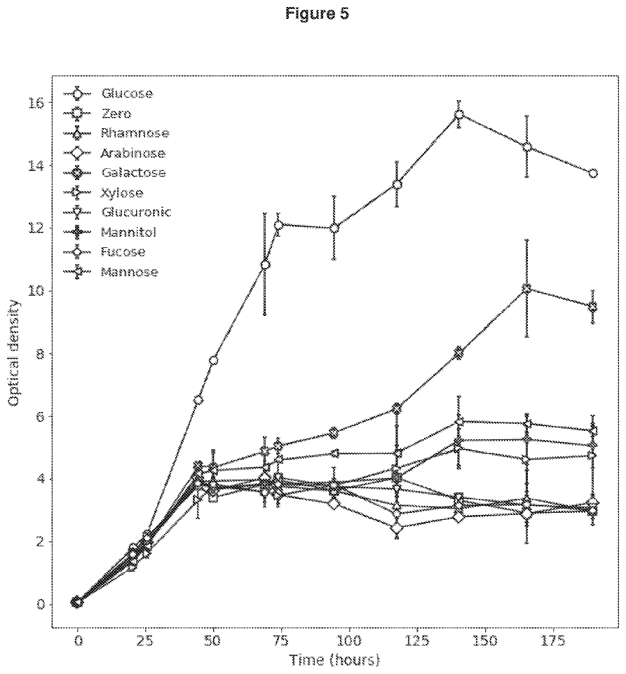

The observed growth curves (FIG. 5) demonstrate strong growth of H. mediterranei on glucose. The second best substrate was galactose. Mannose, mannitol and xylose performed similarly, with a small amount of additional growth seen above control cultures (prepared without sugar substrates). Arabinose, fucose, rhamnose and glucuronic acid did not show any significant difference compared to control cultures, likely indicating that H. mediterranei is unable to utilise these substrates.

The results suggest that both green (Chlorophyta) and red (Rhodophyta) macroalgae are best suitable feedstocks for PHA production using H. mediterranei. The main monosaccharide produced by Ulva sp. (a green seaweed) is glucose, while Gracilaria sp. (red) typically produce primarily galactose followed by glucose.

### Enzymatic Hydrolysis—Enzyme Type and Concentration.

Experimentation with different blends and concentrations of Viscozyme and Celluclast enzyme preparations (Novozymes) was performed to select an enzyme treatment that provides high glucose production while minimising costs. Experiments were prepared by acid pre-treatment of 10% (w/v) solids load of seaweed in an autoclave (25 mM citric acid, 120° C., 2 h). Samples were cooled to 55° C., adjusted to pH 5 with 2 M NaOH and treated with enzyme for 17 h in a shaking water bath (55° C., 130 rpm).

Results (Table 3) demonstrate a high glucose concentration (19.6 g/L) achieved with 0.2 mL/g biomass of a 1:1 blend of Viscozyme and Celluclast. A similar result (20.0 g/L) was obtained with a significantly higher enzyme loading (0.5 mL/g of a 1:3 blend of Viscozyme and Celluclast), indicating that minimal benefit is obtained by increasing enzyme load above 0.2 mL/g. While the use of Viscozyme only at 0.2 mL/g provided a comparable result (19.2 g/L) to the 1:1 blend, a combination of cellulase mixtures with different cellulolytic activities is preferred as seaweed can vary significantly in structural and biochemical composition depending on taxonomy, seasonality, and location.

TABLE 3
Final hydrolysate glucose concentrations
with different enzyme blends.

| Enzyme treatment | Glucose concentration (g/L) |
|---|---|
| 0.5 mL/g Celluclast | 17.8 |
| 0.2 mL/g Viscozyme | 19.2 |
| 0.1 mL/g Viscozyme | 15.0 |
| 0.5 mL/g 1:3 blend (Viscozyme:Celluclast) | 20.0 |
| 0.2 mL/g 1:1 blend (Viscozyme:Celluclast) | 19.6 |

### Enzymatic Hydrolysis—Salinity

Enzymatic hydrolysis of pre-treated biomass was performed in both freshwater and seawater to assess the feasibility of producing seaweed hydrolysate using seawater. Experiments were prepared by acid pre-treatment of 10% (w/v) solids load of dried seaweed (Gracilaria sp.) in either freshwater (0 ppt salinity; prepared by reverse osmosis) or seawater (35 ppt salinity; obtained from Waterman's Bay, Australia) in an autoclave (25 mM citric acid, 120° C., 2 h). Samples were then cooled to 55° C., adjusted to pH 5 with 2 M NaOH and treated with enzyme (0.2 mL/g of 1:1 blend of Viscozyme and Celluclast) for 18 h in a shaking water bath (55° C., 130 rpm).

The experiment demonstrated that the activity of the enzymes is impaired in seawater, with seawater hydrolysis samples achieving 65±2% of the glucose concentration of the freshwater hydrolysis samples.

Pre-Treatment with Strong and Weak Acids

As the choice of acid type and concentration during pre-treatment of biomass can affect the performance of both enzymatic hydrolysis (via damage to biomass structures) and hydrolysate fermentability (via production of inhibitors), the use of both a strong acid (sulfuric acid) and a weak acid (citric acid) for pre-treatment was compared. The performance of enzymatic hydrolysis was assessed by hydrolysate glucose concentration, and fermentability was assessed using shake flask cultures of H. mediterranei.

Experiments were prepared by acid pre-treatment of 10% (w/v) solids load of seaweed in an autoclave (120° C., 2 h) using a 25 mM concentration of either citric acid or sulfuric acid. Samples were cooled to 55° C., adjusted to pH 5 with 2 M NaOH and treated with enzyme (0.2 mL/g of 1:1 blend of Viscozyme and Celluclast) for 24 h in a shaking water bath (55° C., 130 rpm).

Following enzymatic hydrolysis, hydrolysates produced with both citric acid and sulfuric acid had similar glucose concentrations, with citric acid performing slightly better (19.5 g/L vs 18.1 g/L for citric vs sulfuric acid, respectively) at the 25 mM concentration used.

To assess the fermentability of the hydrolysates, the hydrolysates were used to prepare growth media. The hydrolysates were adjusted to pH 7 using 2 M NaOH and then combined with salts to give the composition provide for in ATCC Medium 1176 (Halobacterium medium) shown in FIG. 2 (with the omission of glucose and yeast extract). A final concentration of 1 mL per L of SL-4 trace metals (FIG. 3) was also added to all fermentation media. The media was then diluted at different strengths (33%, 25% and 17%) with a solution containing salts as provided for in ATCC Medium 1176 (FIG. 2; with omission of glucose and yeast extract). Volumes of 60 mL of each dilution were transferred to 250 mL flasks and supplemented with 2.5 g/L yeast extract. The flasks were then inoculated with H. mediterranei (grown from a single colony in ATCC Medium 1176) to an initial optical density (600 nm; OD₆₀₀) of ˜0.5. Flasks were incubated at 40° C. and 150 rpm, and growth was monitored by OD₆₀₀ measurements over a 112 h period.

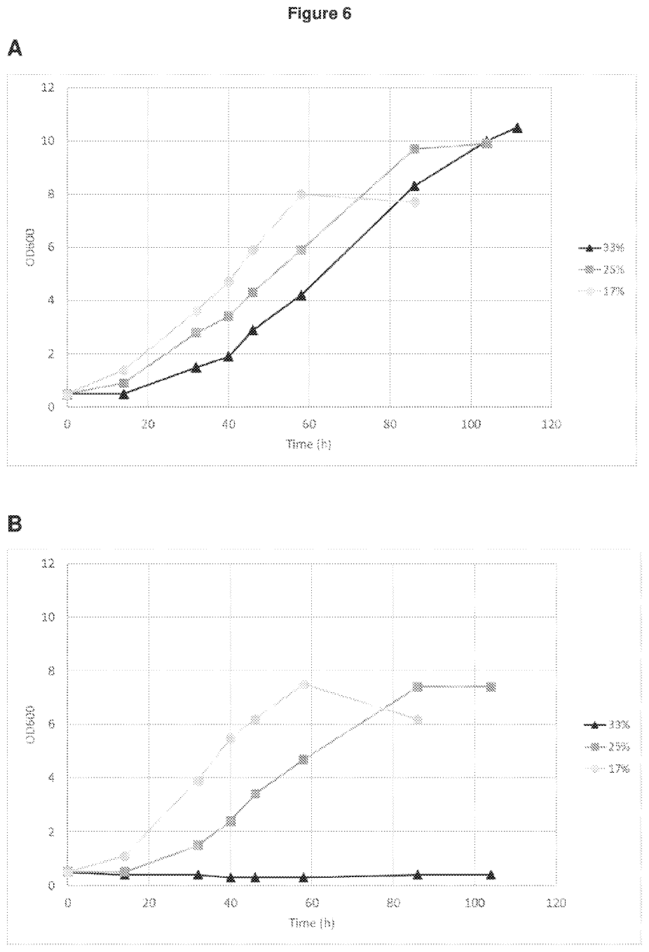

A stark difference in fermentability between citric acid- and sulfuric acid-treated hydrolysates was observed in the hydrolysate media prepared at 33% strength, with the citric acid sample reaching a final OD₆₀₀ of 10.0 while no growth was detected in the sulfuric acid sample at the same dilution (FIGS. 6A and 6B). This suggests the presence high levels of inhibitory compounds (for example, 5-HMF) in the sulfuric acid-treated hydrolysates compared to the citric acid samples. This was further demonstrated by a less significant difference in growth between citric and sulfuric acid samples when hydrolysates were diluted to 25% strength, and no observable difference in growth when the hydrolysates were diluted to 17% strength, indicating that inhibitory compounds were at sub-inhibitory concentrations in the latter dilution.

As both acid types produced hydrolysates with similar glucose concentrations, citric acid pre-treatment appears to result in significantly lower production of inhibitory compounds than sulfuric acid and is a preferred choice.

To confirm production of PHAs using hydrolysate produced with citric acid pre-treatment, PHAs were isolated from the cells using hypo-osmotic shock. Volumes of 50 mL of cell culture were centrifuged (4500 rpm, 25 min, 4° C.) and the supernatant discarded. The cell pellets were then resuspended in 50 mL of a solution (0 ppt) containing 0.1% SDS and agitated to lyse cells. The PHA pellets were collected by centrifugation (4500 rpm, 25 min, 4° C.) and washed twice with de-ionised water. The PHAs were then dried overnight at 60° C. and weighed. The PHA concentrations determined by weighing are presented in Table 4 below.

TABLE 4
PHA concentrations in H. mediterranei cultures grown on
citric acid-treated hydrolysate at different dilutions.

| Dilution | Extracted PHA (g/L) |
|---|---|
| 33% | 1.48 |
| 25% | 1.52 |
| 17% | 1.20 |

### Effects of Yeast Extract and Hydrolysate Dilution

Experiments were performed to investigate the effect of hydrolysate dilution and supplementation with yeast extract on the growth of H. mediterranei. Hydrolysate dilution can lessen the effect of inhibitory compounds on cell growth but also lowers the concentration of sugar and nitrogen present in the hydrolysate, which can limit growth rates and final cell densities. Yeast extract provides a rich source of nitrogen, carbon, and other nutrients but increases costs.

Hydrolysates were prepared as described in previous experiments (pre-treatment at 10% solids load with 25 mM citric acid at 120° C. for 2 h; enzymatic hydrolysis with 0.2 mL/g 1:1 Viscozyme and Celluclast blend; addition of ATCC Medium 1176 salts). Dilutions were then made with a solution containing salts as provided for in ATCC Medium 1176 (without yeast extract or glucose) at 50%, 33%, 25% and 17% strengths and 30 mL volumes were transferred to 150 mL flasks. Two samples were prepared for each dilution, with one receiving 2.5 g/L yeast extract and the other receiving no yeast extract. The flasks were then inoculated with H. mediterranei (grown from a single colony in ATCC Medium 1176) to an initial OD₆₀₀ of ˜0.5. Flasks were incubated at 40° C. and 150 rpm, and growth was monitored by OD₆₀₀ measurements over a 125 h period.

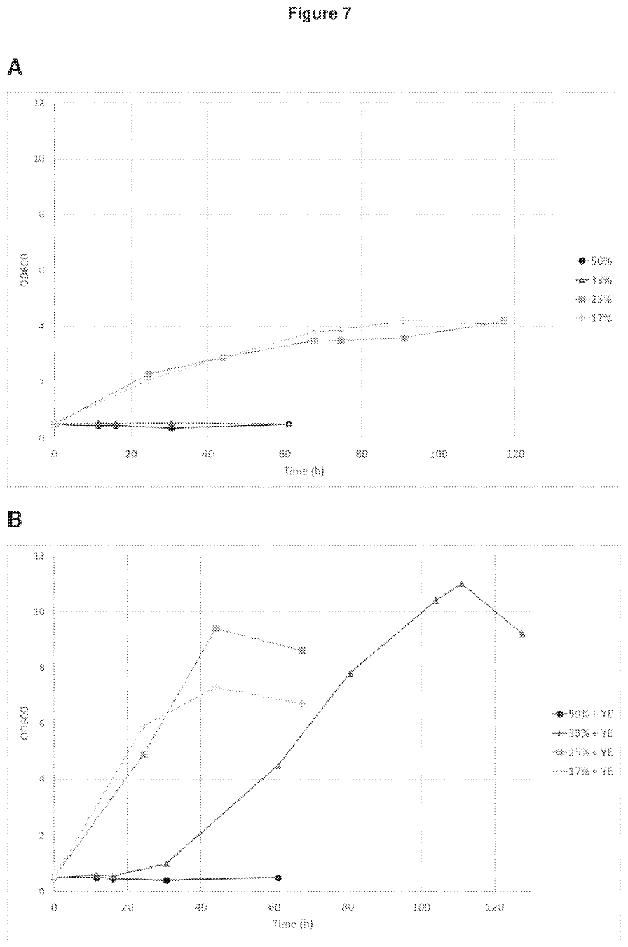

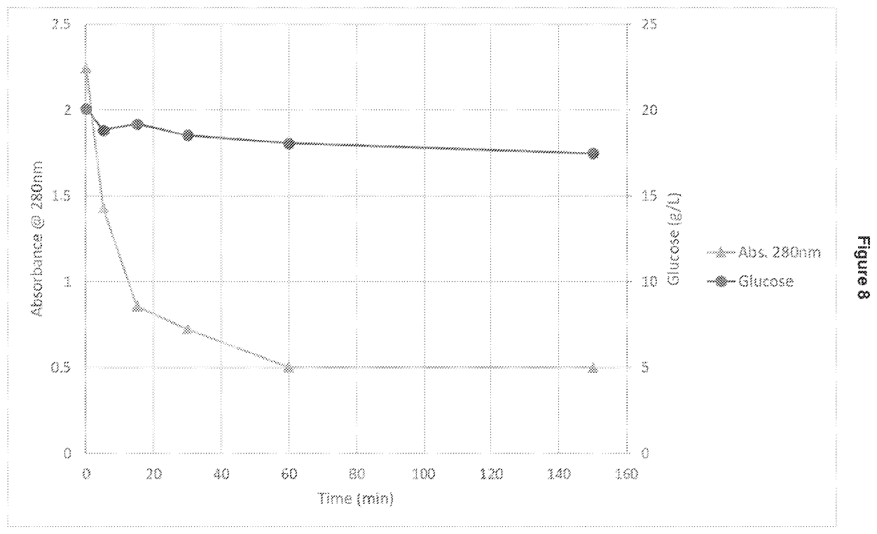

Hydrolysate dilution and yeast extract were observed to have significant effects on cell growth (FIGS. 7A and 7B). At the highest hydrolysate strength of 50%, no growth was observed either with or without yeast extract, likely due to a high level of fermentation inhibitors. At 33% strength, H. mediterranei was only able to grow in the presence of yeast extract, suggesting that yeast extract provided nutrients that lessened the impact of inhibitory compounds. At 25% and 17% strength, cells with yeast extract grew significantly faster compared to the 33% strength, again most likely due to less inhibitors, but reached lower final cell densities, likely due to less glucose at these dilutions. The 25% and 17% strength samples without yeast extract both grew significantly slower and reached lower final cell densities compared to the corresponding samples with yeast extract.

The results demonstrate the significance of inhibitory compounds present in the hydrolysate, either extracted from seaweed (for example, phenolic compounds) or produced during pre-treatment (for example, 5-HMF), on the growth of H. mediterranei. The observation that yeast extract appears to reduce the effects of inhibition is an important result that warrants further investigation.

### Charcoal Treatment

Experiments were performed to assess the effectiveness of activated carbon (charcoal) on the removal of inhibitors such as 5-HMF and phenolic compounds from the hydrolysate. First, hydrolysates were treated with charcoal while monitoring both the solution absorbance at 280 nm (corresponding to phenolic groups) and the glucose concentration (to assess for any loss of sugar during charcoal treatment). The effectiveness of charcoal treatment was then assessed by monitoring the growth of H. mediterranei in shake flask cultures containing growth media prepared with hydrolysate.

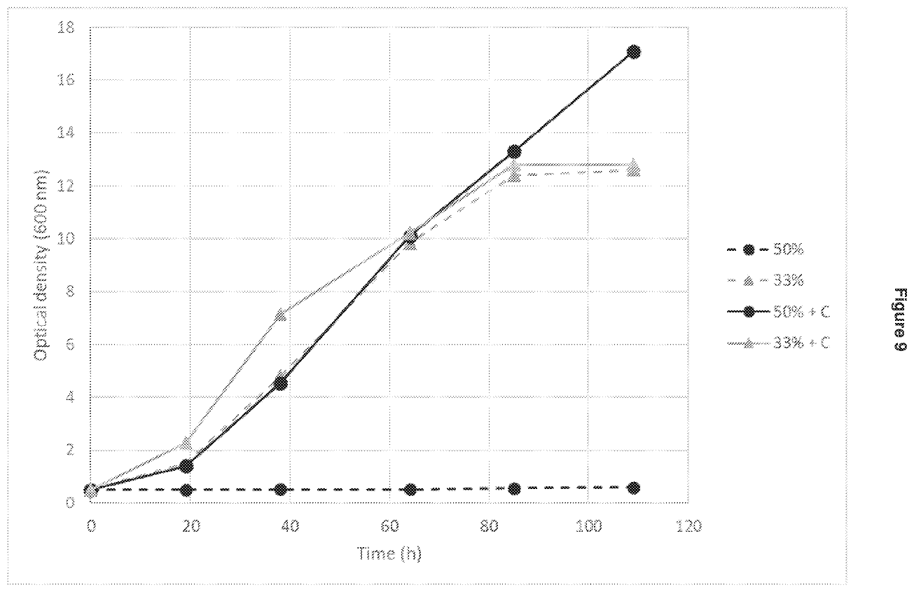

Hydrolysates were prepared as described in previous experiments (pre-treatment at 10% solids load with 25 mM citric acid at 120° C. for 2 h; enzymatic hydrolysis with 0.2 mL/g 1:1 Viscozyme and Celluclast blend). The hydrolysates were then combined with a 2.5% w/v of activated charcoal granules and incubated on a rotary shaker incubator at 150 rpm and 40° C. for 150 min. Samples were removed periodically, centrifuged to remove charcoal, and tested for absorbance at 280 nm (A₂₈₀) and glucose via a blood glucose monitor strip test (Abbott FreeStyle Optium Neo glucose monitor). As shown in FIG. 8, an ˜80% drop in A₂₈₀ was observed over the course of 60 min which then remained stable to 150 min, potentially indicating removal of phenolic compounds from the hydrolysate. The glucose concentration decreased by approximately 14% during the treatment.

Hydrolysates produced with either no charcoal treatment or treatment with charcoal (2.5% w/v) for 60 min were used to prepare growth media by adding ATCC Medium 1176 salts as described previously. Growth media were prepared at 50% and 33% strength and inoculated with H. mediterranei as described previously.

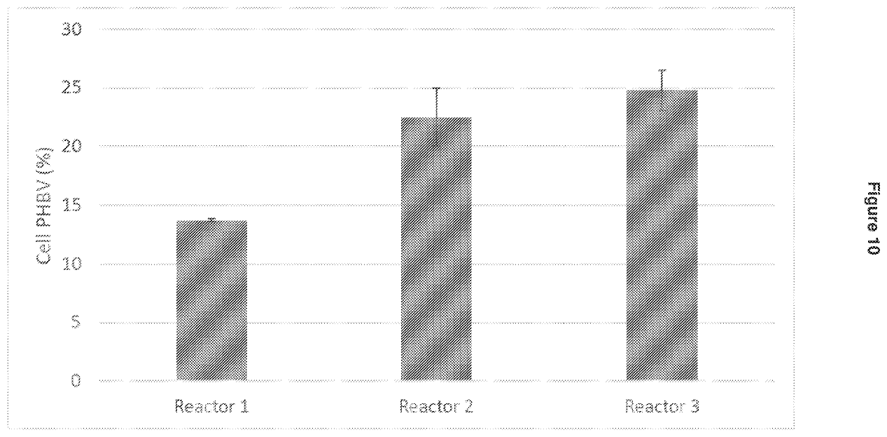

The effects of charcoal treatment were very significant in hydrolysates at 50% strength (FIG. 9). No growth was observed without charcoal treatment, while the charcoal treatment allowed significant growth up to a final OD₆₀₀ of 17.0. The effects of charcoal treatment on the 33% strength medium did not produce a noticeable effect on final cell density but may have provided a slightly faster growth rate in the early stages of the culture.

The results demonstrated that charcoal treatment likely reduced the concentrators of one or more inhibitors below a critical level, allowing growth at a strong hydrolysate strength. This allows media with higher levels of seaweed-derived sugars to be used, providing high final cell densities for PHBV extraction.

### Scaled-Up Hydrolysate Production (7 L)

The production of hydrolysate from Gracilaria sp. was demonstrated at 7 L scale and used to produce growth media for H. mediterranei bioreactor cultures.

A mass of 750 g of freeze-dried, milled seaweed (Gracilaria sp.) was placed in a 10 L hydrolysis reactor (Model: FCF-10L, Zhengzhou Keda Machinery and Instrument Equipment Co. Ltd) and mixed with 7.5 L of 25 mM citric acid solution. The reactor was sealed and heated to a temperature of approximately 120° C., as determined by a gauge pressure of around 100 kPa. The mixture was continuously agitated (200 rpm) by a mechanical stirrer and held at this temperature for 2 h. The reactor was then cooled to 55° C. via internal cooling coils.

The mixture was adjusted to a pH of 5 using 2 M NaOH. Volumes of 75 mL each of Viscozyme and Celluclast (0.1 mL each per g seaweed) were then added. The reactor temperature was maintained at 55° C. with constant agitation (200 rpm) for 20 h. The mixture was then removed from the reactor and centrifuged in batches (4500 rpm, 25 min) to remove solid biomass.

The supernatant was then returned to the reactor and combined with 2.5% w/v of activated charcoal granules. The mixture was agitated for 60 min (200 rpm), removed from the reactor and centrifuged in batches (4500 rpm, 25 min) to remove charcoal and then stored at 4° C. until required for bioreactor culture.

Batches produced by this method resulted in 7 L of liquid hydrolysate with an average glucose concentration of 18.1±1.1 g/L (average of three independent batches).

### Three-Stage Continuous Fermentation System-Synthetic Medium

A three-stage continuous fermentation system was developed and tested for the production of PHAs. The continuous fermentation system was devised to promote growth of H. mediterranei in the first bioreactor, while the second and third bioreactors were primarily for PHA production. A fourth bioreactor was included to act as a cell collection vessel for periodic harvesting of PHAs. Bioreactors 1, 2 and 3 had independent feeds to allow control of medium composition in each reactor.

The three-stage continuous fermentation system was examined using synthetic medium (based on ATCC Medium 1176, FIG. 2). First, the three bioreactors were filled to a volume of 3.5 L with a solution containing salts as provided for in ATCC Medium 1176 (supplemented with 1 mL per L of SL-4 metal mix; FIG. 3) and inoculated to an initial OD₆₀₀ of approximately 0.5 using a shake flask inoculum of H. mediterranei. The bioreactors were run in batch mode for 43 h at 40° C., with pH controlled at a setpoint of 7.0 and dissolved oxygen maintained around 20-40% by stirring speed and aeration rate, until the glucose concentration in each bioreactor was exhausted.

The system was then changed to continuous mode. The total flow rates between the sequential bioreactors and the individual feed flow rates for each reactor are provided in Table 5 below. This table also shows the feed composition for each bioreactor. All feed solutions consisted of with a solution containing salts as provided for in ATCC Medium 1176 with modified glucose and yeast extract concentrations as described in Table 5.

TABLE 5
Flow rates and feed compositions for the three-
stage continuous fermentation system.

| | | | | Total | | Feed |
|---|---|---|---|---|---|---|
| | | Working | Feed | flow rate | Feed | yeast |
| | | volume | flow rate | (out) | glucose | extract |
| Bioreactor | Model | (L) | (mL/min) | (mL/min) | (g/L) | (g/L) |
| 1 | Minifors 2 | 3.5 | 1.45 | 1.45 | 20 | 6 |
| | (Infors HT) | | | | | |
| 2 | 5 L glass | 3.5 | 0.36 | 1.81 | 30 | — |
| | fermenter | | | | | |
| | (Bailun Bio) | | | | | |
| 3 | 5 L glass | 3.5 | 0.36 | 2.17 | 40 | — |
| | fermenter | | | | | |
| | (Bailun Bio) | | | | | |
| 4 | 5 L glass | 0-4 | — | — | — | — |
| | fermenter | | | | | |
| | (Bailun Bio) | | | | | |

The PHAs produced by H. mediterranei in the continuous process were periodically harvested from the fourth bioreactor by the following process. A volume of 2-4 L of fermentation broth was removed from the reactor and centrifuged (10,000 rpm, 15 min) to collect cells. The supernatant was discarded and the cells were resuspended in a solution (0 ppt) containing 0.1% sodium dodecyl sulfate (SDS) of the same volume as the initial fermentation broth harvest. The mixture was agitated with a magnetic stirrer (1100 rpm) for 1 h. The solution was then centrifuged to recover solid PHAs and the supernatant was discarded. The crude PHAs were then resuspended in de-ionised water at the same volume of the previous extraction step. The mixture was again stirred for 1 h before centrifugation and discarding of the supernatant. The wash procedure was performed two times. The washed PHAs were then dried for 24 h at 60° C. in a vacuum oven.

Results of PHA harvests performed on five consecutives days of continuous culture are shown in Table 6 below. The results demonstrate consistent PHA yields across five days of operation of the system.

TABLE 6
PHA harvest yields from the three-stage continuous culture system.

| | | PHBV | PHBV |
|---|---|---|---|
| Harvest | Volume (L) | mass (g) | concentration (g/L) |
| 1 | 2.7 | 5.42 | 2.01 |
| 2 | 2.7 | 5.80 | 2.15 |
| 3 | 2.7 | 6.90 | 2.56 |
| 4 | 2.7 | 5.81 | 2.15 |
| 5 | 2.8 | 5.48 | 1.96 |

The amount of PHBV as a proportion of cell weight was assessed throughout the run using GC-MS. Typically, this increased from first bioreactor to the later bioreactors, indicating increasing intracellular accumulation of PHBV. The proportion of PHBV at a selected timepoint (121 h) is shown in FIG. 10.

### Three-Stage Continuous Fermentation System-Gracilaria Hydrolysate

The three-stage continuous bioreactor system described in the previous experiment was adapted for PHA production with hydrolysate produced from Gracilaria seaweed. The hydrolysate for the experiment was produced by multiple 7 L batches as described above.

The three-stage reactor system was performed essentially as before, with reactor feeds consisting of diluted hydrolysate (in ATCC Medium 1176 salts; FIG. 2). The flow rates and feed compositions are provided in Table 7 below.

TABLE 7
Flow rates and feed compositions for the three-stage continuous
culture system using Gracilaria hydrolysate.

| | | | | Total | | Feed |
|---|---|---|---|---|---|---|
| | | Working | Feed | flow rate | Feed | yeast |
| | | volume | flow rate | (out) | hydrolysate | extract |
| Reactor | Model | (L) | (mL/min) | (mL/min) | strength(%) | (g/L) |
| 1 | Minifors 2 | 3.5 | 1.45 | 1.45 | 50 | 2.5 |
| | (Infors HT) | | | | | |
| 2 | 5 L glass | 3.5 | 0.36 | 1.81 | 75 | — |
| | fermenter | | | | | |
| | (Bailun Bio) | | | | | |
| 3 | 5 L glass | 3.5 | 0.36 | 2.17 | 100 | — |
| | fermenter | | | | | |
| | (Bailun Bio) | | | | | |
| 4 | 5 L glass | 0-4 | — | — | — | — |
| | fermenter | | | | | |
| | (Bailun Bio) | | | | | |

Over the course of the continuous fermentation process, agar dissolved in the Gracilaria hydrolysate began to precipitate and adhere to the inside of the bioreactor, as well as cause blockages of the transfer tubes between reactors. The precipitated agar also significantly interfered with biomass measurements (both by optical density and dry weight measurements). The precipitated agar was also a significant contaminant of harvested PHAs, preventing accurate mass measurements.

The results of this experiment suggest that a hydrocolloid (agar) extraction step from the seaweed (Gracilaria) biomass should be considered prior to hydrolysate production.

Hydrolysate Production with Green Seaweed (Ulva Sp.)

As green seaweed (Chlorophyta) was identified as a potentially suitable feedstock for PHBV production by H. mediterranei, experiments were performed to produce hydrolysate from Ulva sp. and use this as a feed for a single-stage continuous fermentation system.

The Ulva hydrolysate was performed by hydrothermal and enzymatic hydrolysis in a 10 L hydrolysis reactor. The Ulva biomass was received as a dry powder. A mass of 700 g of the powder was combined with 7 L deionised water and heated to a temperature of 150° C. It was held at this temperature with constant agitation (200 rpm) and then cooled to 50° C. The mixture was adjusted to pH 5 using 2 M NaOH and then 700 mL of Viscozyme (1.0 mL/g seaweed) was added. The mixture was incubated for 24 h and then separated by centrifugation. The liquid hydrolysate fraction was adjusted to pH 7 with 2 M NaOH, then diluted to 50% strength with water and salts to give the composition of ATCC Medium 1176 (FIG. 2; without addition of glucose or yeast extract). A volume of 1 mL per litre of SL-6 metals mix was also added (FIG. 3).

The growth medium was stored at 4° C. until required for continuous fermentation experiments.

### Single-Stage Continuous Fermentation System-Ulva Hydrolysate

A volume of 3.5 L of 50% strength growth medium produced with Ulva as described above was added to a bioreactor (Minifors 2; Infors HT).

The bioreactor was heated to 40° C. and then inoculated with H. mediterranei to give an approximate initial OD₆₀₀ of approximately 0.5. The bioreactor ran in batch mode for 36 h at 40° C. with pH controlled at a setpoint of 7.0 and dissolved oxygen maintained around 20-40% by stirring speed and aeration rate.

The bioreactor was then fed with 50% strength Ulva growth medium at a rate of approximately 1.93 mL/min using a peristaltic pump (0.033 h⁻¹ dilution rate). Fermentation broth was removed from the bioreactor by a peristaltic pump at the same rate and transferred to a 5 L Erlenmeyer flask agitated by a magnetic stirrer. The collected broth was harvested daily for PHAs by water extraction as described for previous experiments.

The harvests demonstrated consistent PHA production over a six-day period as shown in Table 8 below.

TABLE 8
PHA harvest yields from the single-stage continuous
fermentation system using Ulva hydrolysate.

| | | PHA | PHA concentration |
|---|---|---|---|
| Harvest | Volume (L) | mass (g) | (g/L) |
| 1 | 3.0 | 6.25 | 2.08 |
| 2 | 2.5 | 8.16 | 3.26 |
| 3 | 2.5 | 9.20 | 3.68 |
| 4 | 2.8 | 8.60 | 3.07 |
| 5 | 2.6 | 6.40 | 2.46 |
| 6 | 4.0 | 7.50 | 1.88 |

Comparing Upstream Process Efficiencies when Using Seawater Compared to Freshwater

Experiments were conducted to investigate the effect of using seawater rather than freshwater for agar extraction and production of a glucose-rich hydrolysate from seaweeds.

Specifically, the following parameters were investigated:
Agar yield, in grams of agar per 100 g of dry seaweed biomass;
Agar purity, in product clarity level as assessed via visual inspection of samples;
Hydrolysis glucose yield, in grams of glucose per 100 g of dry seaweed biomass;
Inhibitor formation during acid pre-treatment, in grams of 5-hydroxymethylfurfural (5-HMF) per litre of hydrolysate; and
Inhibitor removal via detoxication (over-liming and charcoal pre-treatments), in grams of 5-HMF removed.

### Methods

Agar extraction. 300 g of dried red seaweed (Gracilaria sp. purchased from Indonesia) were milled using a food processor (Robot Coupe Blixer® 7; particle size of <2 mm), and then split it into 6 sub-samples (50 g each); with each soaked in 1000 ml of water (1:20 w/v) at ambient temperature for 3 h. Out of these 6 samples, 3 were soaked in seawater (35 ppt salinity) and 3 in freshwater (0 ppt salinity). Both seawater and freshwater were locally sourced in Western Australia (31.8521° S, 115.7518° E). The freshwater was tap water filtered via deionisation and reverse osmosis (BOSS 031-4P system), while seawater was obtained via the ocean pumps and filtration systems of an aquaria facility (Indian Ocean Marine Research Centre Watermans Bay, Western Australia).

The seaweed-water mixtures were heated to 95° C. in a water bath, held at this temperature for 3 h, and then centrifuged (Himac CR-3ONX, R9A2-4234 rotor; 8500 rpm, 10 min) for separation of the liquids containing agar from the solids. The solids were washed with 300 mL of hot (95° C.) water (either seawater or freshwater; same as used for first wash/agar extraction) and centrifuged again (8500 rpm, 10 min). The washed solids were set aside (for later enzymatic treatment and hydrolysate production), while the liquid fractions were combined with the first liquid fractions (containing the agar extracts) and then allowed to gel at room temperature overnight. The gels were frozen at −20° C., thawed at room temperature and then dried in an oven at 60° C. for 48 h. The dried agar was washed with 1 L cold (4° C.) freshwater on a magnetic stirrer for 1 h, filtered through muslin cloth, and then washed again under the same conditions (1 L 4° C. water) and dried at 60° C. for a further 24 h. To remove remaining water, the agar was dehydrated by soaking in acetone (50 mL) for 1 h and drying at 60° C. for a further ˜2.5 h. The resulting dry agar were visually inspected to assess clarity level (directly proportional to purity level), and agar yields were quantified gravimetrically as grams of dried agar per 100 g of dried seaweed biomass.

Acid Pre-treatment. Following agar extraction, a pre-treatment process was applied to the solids to make cellulose more accessible to enzymatic digestion. Twelve samples of 60 g wet solids were combined with 30 ml of water (either seawater or freshwater, as shown in Table 9) containing 430 mg citric acid (to give a final concentration of 25 mM). These seaweed-water mixtures were heated to 120° C. in an autoclave (Biobase BKQ-B75I) for 30 min, cooled to ˜50° C. in a water bath (ZZKD) and separated by centrifugation (Eppendorf 5910R; 4500 rpm, 20 min). The solids were stored until further use, while the liquid fractions had their 5-HMF levels measured with a HMF test kit (Merck®), and then submitted to a detoxication process prior to re-mixing with the solids for the enzymatic treatment (see details below).

TABLE 9
Type of water used for agar extraction and hydrolysate production.
The second column indicates the water type used for agar extraction,
while the third column shows the water type used for the hydrolysis
process (i.e. acid pre-treatment, detoxication steps and enzymatic
treatment). The samples included: (1) three samples with full
processing using freshwater; (2) three samples with freshwater
agar extraction and seawater hydrolysis; (3) three samples with
seawater agar extraction and freshwater hydrolysis; (4) three
samples with full processing with seawater, to give a total
of 12 seaweed biomass samples.

| | Agar Extraction | Hydrolysis | No. of Samples |
|---|---|---|---|
| 1 | Freshwater | Freshwater | 3 |
| 2 | Freshwater | Seawater | 3 |
| 3 | Seawater | Freshwater | 3 |
| 4 | Seawater | Seawater | 3 |

Detoxication Pre-treatment. Since the relatively mild acid pre-treatment used (120° C., 25 mM citric acid) generates very low levels of glucose (<0.1 g/L), it is possible to perform a detoxication process prior to enzymatic treatment to remove fermentation inhibitors such as phenolic compounds and 5-HMF without the risk of glucose loss. As such, the inventors developed a detoxication process with two treatments (see details below). The first, called over-liming treatment afterwards, involved adjusting the liquid fractions pH from ˜3.5 to 11 using a slurry of calcium hydroxide (All Chemical, Australia) and water (1:1 w/v), agitating the solutions for 30 min on a rotary shaker (Thermo Scientific MaxQ 8000 shaker incubator; 40° C., 150 rpm) and then removing the solids via centrifugation (Eppendorf 5910R; 4500 rpm, 20 min). The second, called charcoal treatment afterwards, consisted of adjusting the post over-liming liquids to pH 5 with 2 M HCl and then combining with 2.5% w/v of activated charcoal powder (All Chemical, Australia), agitating the mixtures for 30 min as previously, and centrifuging (as before) to remove the charcoal. Detoxification efficiency was assessed by measuring the concentration of 5-HMF using HMF test kits (Merck®).

Enzymatic Treatment. The pre-treated supernatants were recombined with their respective solids (as obtained after acid pre-treatment) and then exposed to an enzymatic hydrolysis process aimed at breaking down cellulose into glucose. Two cellulolytic enzyme preparations-Celluclast and Viscozyme-were added (0.1 mL each per g of dry seaweed biomass), and the mixtures incubated at 50° C. and 130 rpm in a water bath for 20 h. Finally, the mixtures were separated by centrifugation (4500 rpm, 20 min) to separate the spent seaweed biomass (solids) from the hydrolysates (liquids). Both the salinity (in parts per thousand (ppt), using an EcoSense EC300 salinity meter) and glucose concentration (in g/L, using D-glucose HK assay kit, Megazyme®) of the hydrolysate was measured.

### Results & Discussion

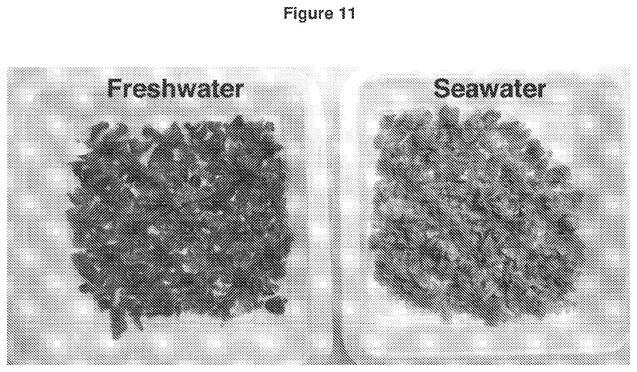

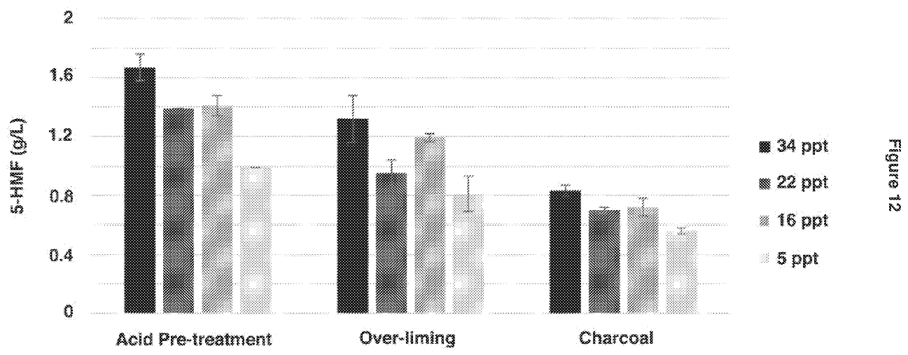

Agar Yields and Quality. Average agar extraction yields were equal to 22.7 g per 100 g of dry seaweed biomass±1.7 standard deviation (std) when using freshwater, and 16.5 g per 100 g of dry seaweed biomass (±0.5 std) when using seawater. Even though the use of seawater resulted in a 6% reduction in agar yields, the seawater-based product was lighter in colour, likely due to a higher purity level (FIG. 11). This means the seawater-based product may have higher market value and/or require less downstream processing (for example, bleaching or further purification) to produce a market-ready and more sustainable product.

Glucose Yields. Average glucose yields obtained via seawater-based hydrolysis and freshwater-based hydrolysis were comparable but had a negative correlation with medium salinity (Table 10). This is likely due to impaired cellulase performance at higher salt concentrations.

TABLE 10
Average glucose yields (in g of glucose per 100 g of dry
seaweed biomass), glucose concentrations (in g of glucose
per L of hydrolysate), and medium salinity (in ppt) under
different schemes of seawater and freshwater usage.

| | | Glucose | Glucose | |
|---|---|---|---|---|
| Agar | | Yield | Concentration | Salinity |
| Extraction | Hydrolysis | (g/100 g) | (g/L) | (ppt) |
| Freshwater | Freshwater | 15.4 ± 0.7 | 17.4 ± 1.0 | 5 |
| Freshwater | Seawater | 14.9 ± 0.5 | 16.6 ± 0.6 | 16 |
| Seawater | Freshwater | 13.5 ± 1.3 | 18.1 ± 1.8 | 22 |
| Seawater | Seawater | 12.4 ± 0.7 | 16.7 ± 1.0 | 35 |

Fermentation Inhibitor Formation and Removal. Levels of the fermentation inhibitor 5-HMF following acid pre-treatment grew proportionally to salinity level in the medium (FIG. 12). This suggests higher salt concentrations lead to faster rates of polysaccharide degradation, meaning shorter pre-treatment times, milder temperatures and/or lower acid concentrations may reduce 5-HMF production in the presence of seawater.

Both over-liming and charcoal treatments contributed to 5-HMF removal, with the combination of both steps reducing the inhibitor levels by 50% regardless of salinity level (see FIG. 12). Inventors note that the different salinity levels (as formed via the use of seawater and/or freshwater; see Table 9) affect the level of 5-HMF formed during acid pre-treatment, but not the detoxication efficiency of over-liming or charcoal treatment.

### Testing the Inventive Seawater-Based Process at Pilot Scale

Experiments were conducted to:
Test seawater upstream process efficiency at ˜20 L scale.
Assess effects of hydrolysate dilution (100%, 50% strength) and yeast extraction concentration (0, 0.3, 1, 2 g/L) on microbial growth and PHA yields.
Demonstrate a semi-continuous production of PHAs using a 4 L capacity bioreactor.

### Methods

Agar extraction. The upstream seawater-based process developed at bench scale in the previous experimentation (Comparing upstream process efficiencies when using seawater compared to freshwater), consisting of agar extraction, acid pre-treatment, detoxification, and enzymatic treatment, was scaled up to produce ˜20 L of hydrolysate for PHA production via saltwater fermentation (see details in the next sections, under ‘thalassic fermentation’).

2 kg of dried red seaweed (Gracilaria sp. purchased from Indonesia) was milled using a food processor (Robot Coupe Blixer® 7; particle size of <2 mm), and then soaked in 40 L of seawater overnight at ambient temperature.

This seaweed-seawater mixture was heated to 100° C. for 2 h, inside a 50 L capacity double-walled glass reactor fitted with a mechanical agitator (Model: S-50L, Zhengzhou Keda Machinery and Instrument Equipment Co. Ltd), and using thermal fluid (Duratherm 450, Duratherm Fluids, USA) in a recirculating heater (Model: GDX-50L-30C, Zhengzhou Keda Machinery and Instrument Equipment Co. Ltd), with constant agitation at 200 rpm. The mixture was then cooled to approximately 80° C. prior to agar separation via filtration using muslin cloth. The removed solids were washed with approximately 8 L of hot (80° C.) seawater to further remove agar.

Acid Pre-treatment. The wet solids mass weighing ˜10 kg was split into two batches of 5 kg for acid pre-treatment.

Each batch was combined with 2.7 L of seawater and 34 g citric acid in a 10 L capacity stainless-steel hydrolysis reactor (Model: FCF-10L, Zhengzhou Keda Machinery and Instrument Equipment Co. Ltd) fitted with an electric heating element, mechanical stirrer and cooling coils (ZZKD, China). The reactor was heated to 120° C. with constant agitation (300 rpm) until the gauge pressure of the reactor reached 1 bar. The mixture was held at this pressure and agitation for 20 min and then cooled via the internal cooling coils containing a 30% v/v glycol/water mixture recirculated through a chiller unit (Kegland IceMaster G40). The two pre-treated batches were combined and filtered through muslin cloth to separate solids. The solids were washed with approximately 4 L of hot (80° C.) seawater which was combined with the liquid fraction obtained previously.

Detoxication Pre-treatment. The combined liquid had its pH adjusted from ˜3.5 to 11 using a slurry of calcium hydroxide and water and incubated at room temperature for 30 min with occasional manual stirring. The mixture was then filtered through a bed of perlite to remove precipitated solids.

The pH of the recovered liquid was adjusted to 5 using a 2 M HCl solution, and mixed with approximately 2.5% w/v mass of powdered charcoal and again incubated at room temperature for 30 min with occasional manual stirring. The charcoal mixture was then centrifuged in batches (Himac CR-3ONX, R9A2-4234 rotor; 8500 rpm, 15 min) to separate charcoal solids.

Enzymatic treatment. The liquid fraction was re-combined with the seaweed biomass and transferred to the 50 L glass reactor (same as used for agar extraction) and heated to 50° C. A volume of 200 mL Celluclast and 200 ml Viscozyme (Novozymes) were added and the mixture was incubated at 50° C. with constant stirring (200 rpm) for 18 h. The enzyme-treated mixture was removed from the reactor and centrifuged in batches (Himac CR-3ONX, R9A2-4234 rotor; 8500 rpm, 15 min) to remove solids. We've measured both glucose concentrations (in g/L, using D-glucose HK assay kits; Megazyme®) and 5-HMF levels (in g/L using HMF test kit; Merck®) in the final liquid hydrolysate.

Fermentation medium preparation. The liquid hydrolysate fraction (35 ppt salinity) was adjusted to pH 7 with 2 M NaOH and combined with the following amounts of salts per L (adapted from ATCC Medium 1176 formulation to reach a final salinity of 170 ppt): NaCl, 124 g; MgCl₂·6H₂O, 16 g; CaCl₂). 2H₂O, 0.8 g; KCl, 3 g; NaHCO₃, 0.16 g; NaBr, 0.4 g. Trace metals were supplied with the addition of 1 mL per L of SL-6 trace metals mix (FIG. 3) This medium was then chilled overnight to 4° C. and centrifuged in batches (8500 rpm, 15 min, 4° C.) to remove any remaining precipitate.

Fermentation—shake flask experiment. The seaweed-based growth media prepared above (170 ppt salinity, pH 7) was assessed for fermentability and PHA production via a shake flask experiment containing hydrolysate at different dilutions (50% vs 100% strength) and various levels of supplemental yeast extract (0, 0.3, 1 and 2 g of yeast per L of growth medium). For diluted media, dilutions were made with a solution containing salts as provided for in ATCC Medium 1176 (FIG. 2; without glucose or yeast extract) containing 1 mL per L of SL-6 trace metals mix (FIG. 3).

A total of 16 shake flask cultures were prepared, each measuring 30 mL in volume in 150 ml capacity Erlenmeyer flasks. The four levels of yeast extract described above were each tested in both 50% and 100% strength hydrolysate, with two replicates per set of conditions.

The inoculum for this fermentation experiment was prepared by growing a single colony of Haloferax mediterranei (ATCC 33500) in a 50 mL Falcon tube containing 5 mL of a solution containing salts as provided for in ATCC Medium 1176 (FIG. 3) at 40° C. and 150 rpm for 24 h (Thermo Scientific MaxQ 8000). After that, this 5 mL culture was transferred into 250 mL flask containing 45 mL of the same medium and cultured for a further 72 h, under the same fermentation conditions. The 50 mL culture was centrifuged (Eppendorf 5910R; 4500 rpm, 20 min, 4° C.) and the cells were resuspended in salt solution (based on ATCC Medium 1176 without yeast extract or glucose). The cell suspension was used to inoculate the 16 seaweed growth media cultures to a starting optical density (600 nm wavelength (OD₆₀₀); measured with Eppendorf Kinetic® BioSpectrometer) of ˜0.5.

The cultures were incubated at the same conditions as the inoculum (40° C., 150 rpm), with growth assessed every 12-24 h by OD₆₀₀ measurements. Cells were harvested by centrifugation (Eppendorf 5910R; 10100 rpm, 20 min, 4° C.) after growth had ceased or all glucose had been consumed (assessed using an Abbott FreeStyle Optium Neo glucose monitor) with final polyhydroxyalkanoate (PHA) concentrations determined by GC-MS (using same method as described below for monitoring of our semi-continuous fermentation process in a bioreactor).

Fermentation—semi-continuous production in 4 L bioreactor. The inventors demonstrated a thalassic, continuous fermentation system operating semi continuously in a 4 L bioreactor to produce PHAs (Infors Minifors 2®), with an operational volume of 3.5 L. A culture of H. mediterranei was grown in the optimal seaweed-based growth medium identified through the flask experiment described above, namely a 50% dilution of the hydrolysate (diluted with modified ATCC Medium 1176 as described above) with the addition of 2 g/L yeast extract.

Semi-continuous fermentation was carried out by removing 80% (2.8 L) of the operational volume from the bioreactor and replacing it with fresh seaweed-based growth medium every ˜24 h. A total of five such harvests were made over a 138 h period. The removed fermentation broth was used for PHAs extraction as described below.

Inoculation and operating conditions. The bioreactor was autoclaved (121° C., 20 min; Biobase BKQ-B75l) and 3.4 L of growth medium (50% dilution of hydrolysate medium with 2 g/L yeast extract) was added. The bioreactor was heated to a temperature of 40° C. prior to inoculation. For inoculation, a 50 mL culture of H. mediterranei was prepared as described above for shake flask experiments. This was then added to 180 mL of fresh solution ith a solution containing salts as provided for in ATCC Medium 1176 in a 1 L Erlenmeyer flask and incubated (40° C., 150 rpm) for a further 72 h. A volume of 140 mL of this culture was centrifuged (4500 rpm, 20 min) and the cells were resuspended in 100 ml of salt solution as previously described. The resuspended cells were added to the medium in the bioreactor to give an initial OD₆₀₀ of ˜0.5. Throughout fermentation, the temperature was controlled at 40° C. and pH maintained at 7 by automatic addition of 2 M HCl and 2 M NaOH. Dissolved oxygen was maintained at 40% by cascade control of stirrer speed (450-800 rpm) and airflow rate (0-8 L/min). Foaming was controlled by automatic addition of antifoam (Anti-foam 2010; ChemSupply, Australia). Cell growth was monitored by an online turbidimeter (CGQ Sensor BIOR, Aquila Biolabs). Samples were removed at 4-12 h intervals for analysis of biomass, glucose, PHA and nitrogen as described below. A peristaltic pump (Longer G100-1J) was used to remove and replace fermentation broth at harvest.

Biomass monitoring. The cell biomass concentration (in g/L) in the culture broth was measured as ash-free dry weight every ˜12 h throughout the fermentation process. Volumes of 10 mL of fermentation broth were centrifuged and the supernatant discarded. Cell pellets were frozen for at least 1 h at −80° C. and then transferred to a freeze-dryer (Biobase BK-FD10S) and lyophilised for 48 h. Freeze-dried biomass was transferred to a crucible, weighed and then incinerated in a furnace (ThermoFisher M104) at 400° C. for 3 h. Incinerated biomass was cooled and then weighed again to determine ash-free dry weight.

PHA monitoring. The PHBV concentration (in g/L) in the culture broth was measured via GC-MS every ˜12 h throughout the fermentation process. Volumes of 7 mL of fermentation broth were centrifuged (Eppendorf 5910R; 10100 rpm, 20 min, 4° C.), and the cell pellets lyophilized as described above. Freeze-dried cell pellets were weighed and then subjected to methanolysis with 2 mL of 15% sulfuric acid in methanol and 2 mL of chloroform at 100° C. for 2 h 20 min. The chloroform phase containing hydroxyalkanoate methyl esters was then removed for analysis via GC-MS, using benzoic acid as an internal standard. The butyric and valeric methyl esters were evaluated by gas chromatography coupled with mass spectrometry (QP2010; Shimadzu) with a ZB-Wax capillary column (30 m length, 0.25 mm diameter and 0.25 μm thickness). 1 μL sample was injected at 250° C. using a split of 20 and helium as a gas carrier. The temperature increased from 60° C. to 250° C. in a rate of 20° C. per minute, and it was kept at 250° C. for 5 min. Quantification was performed by comparison to a standard curve prepared with PHBV of known purity and composition.

Glucose and nitrogen monitoring. The glucose and total nitrogen concentrations (in g/L) of in the culture broth were measured every ˜12 h throughout the fermentation process. Glucose concentration were determined using D-Glucose (HK) kit (Megazyme®), following the manufacturer instructions.

Total nitrogen concentrations were determined via the persulphate digestion method using a Hach DRB200 digester, and a Hach DR3900 spectrophotometer, with the accompanying Hach proprietary reagents.

PHA Extraction. PHA extraction from harvested H. mediterranei biomass was performed by cell rupturing/lysis via hypoosmotic shock. Volumes of approximately 2.8 L of fermentation medium were removed from the bioreactor and the cells were separated from the broth by centrifugation (Himac CR-3ONX, R9A2-4234 rotor; 8500 rpm, 25 min, 4° C.). The cell pellets were then resuspended in reverse-osmosis water (0 ppt salinity, 0 total dissolved solids; BOSS 031-4P system) containing 0.1% w/v sodium dodecyl sulphate (SDS). Resuspension of the pelleted biomass in the 0.1% SDS solution was such that the final mixture contained approximately 1.1% w/v of H. mediterranei biomass to ensure proper lysis. Proper dispersion of the pelleted cells in the SDS water mix was achieved via vigorous agitation using a magnetic stirrer unit at approximately 1000 rpm rotation speed. Agitation was performed for 1 h until complete lysis of the cells. After this, the lysed cells and SDS solution mix was centrifuged (8500 rpm, 25 min, 4° C.), pelleting crude PHA particles. The PHA pellets were resuspended in the same volume of SDS solution from the lysis stage to wash off any residual cell debris. This wash was performed by agitation using a magnetic stirrer at 1000 rpm for 30 min. After the wash, the PHA particles were pelleted from the mix by centrifugation as above. After this, a final wash by resuspension in freshwater without SDS (equal volume as the previous washes) was performed by agitation with a magnetic stirrer at 1000 rpm for 30 min. A final pelleting step by centrifugation (8500 rpm, 25 min) was then performed to obtain PHA granules. The extracted PHA granules were dried in a vacuum oven for 24 hours at 60° C.

### Results and Discussion

Hydrolysate Production. 21 litres of seaweed hydrolysate was successfully produced out of 2 kg of dry seaweed.

This hydrolysate had 12.0 g of glucose per L of hydrolysate, which is equivalent to 12.6 g of glucose produced per 100 g of dry seaweed. Such glucose yield is comparable to those obtained at bench-scale during the previous experimentation (Comparing upstream process efficiencies when using seawater compared to freshwater) (average±std=12.4±0.7 g per 100 g), indicating good scalability of our hydrolysate production process. The glucose concentration (12.0 g per L), however, was lower than those obtained at bench-scale in the previous experimentation (Comparing upstream process efficiencies when using seawater compared to freshwater) (16.7±1 g/L) due to a higher dilution resulting from process differences in solid/liquid separation, as some separations were performed via filtration in the 20 L process and required additional water to displace liquid from the filter bed.

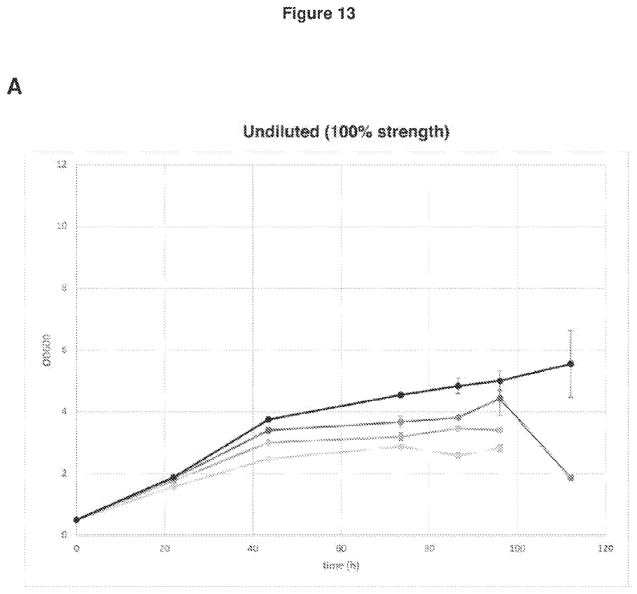

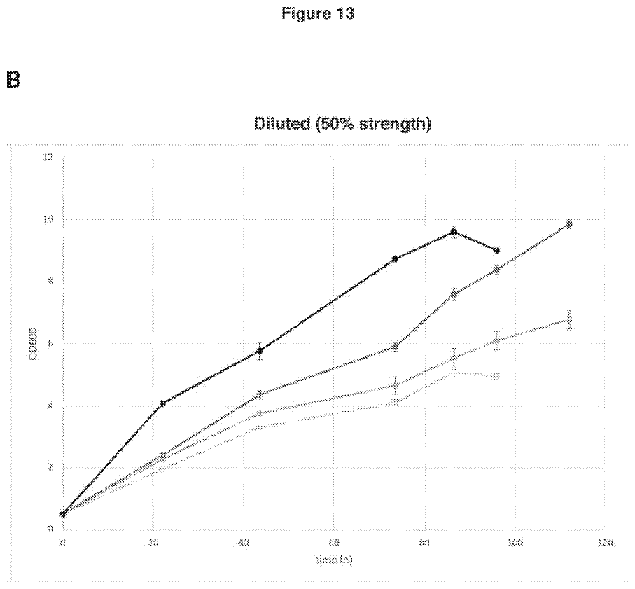

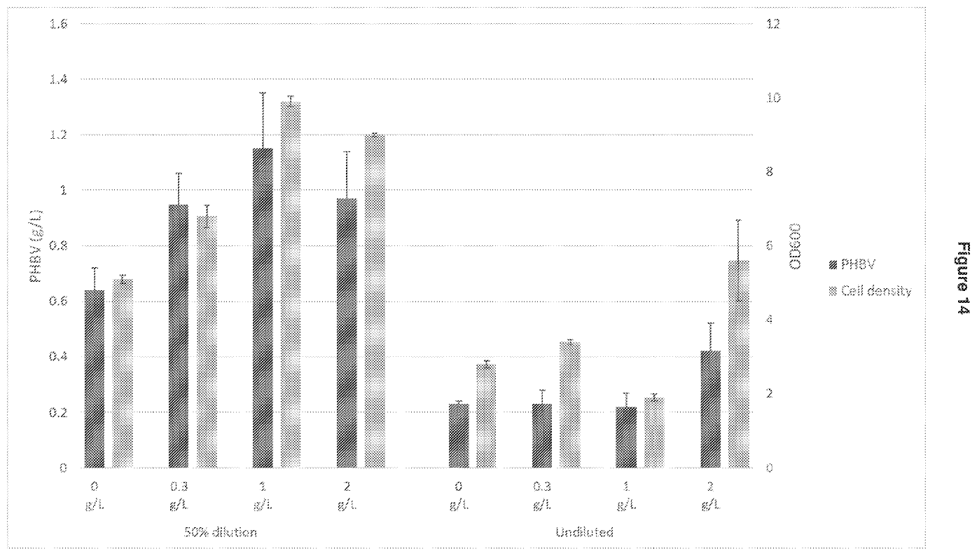

Fermentation—shake flasks experiment. Cell growth was observed in both diluted and undiluted medium, but growth in diluted medium (50% v/v) was significantly stronger (FIG. 13). This is most likely due to inhibitory compounds present in the hydrolysate. As the concentration of 5-HMF in the hydrolysate was relatively low (0.27 g/L), the inhibition may have been due to the presence of other inhibitory compounds present in the seaweed rather than produced during hydrolysis. Potential inhibitors include plant metabolites with antimicrobial activity (for example, flavonoids and other phenolic compounds) or heavy metal ions.

The addition of yeast extract enhanced growth rate and final cell density in both diluted and undiluted medium (see FIG. 13). Yeast extract is a complex substrate that supplies carbon, nitrogen, vitamins, and other growth factors to the fermentation medium. Given that nitrogen and glucose were higher in the undiluted medium compared to the diluted medium, it is unclear whether the growth enhancement seen with yeast extract is primarily due to improved nitrogen or carbon supply, or if it is more related to specific compounds (for example, amino acids, cofactors, nucleotides) that can improve cell health and resilience in the presence of growth-inhibitory compounds in the hydrolysate.

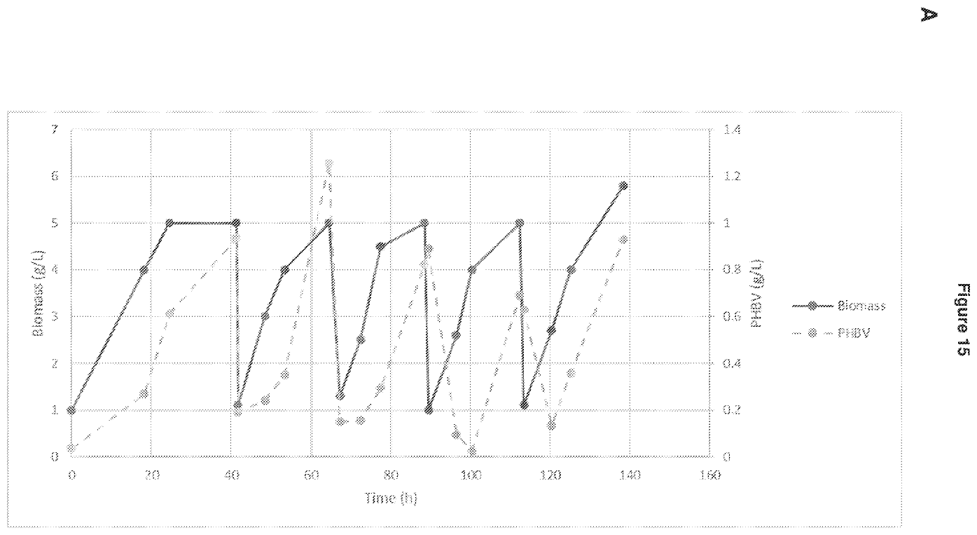

The final medium PHBV concentration was proportional to cell density, varying between 0.4 and 1.2 g PHBV per L of growth media (FIG. 14). Concentrations of PHBV in the undiluted media were significantly lower (0.27±0.10 g/L) than those in diluted medium (0.93±0.21 g/L) due to poorer cell growth as discussed above.

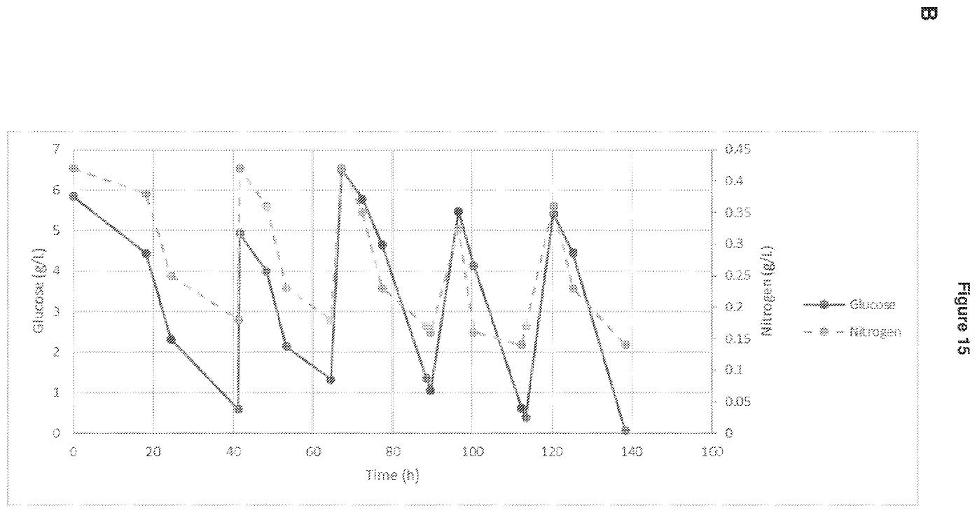

Fermentation—4 L bioreactor, continuous fermentation system. Biomass growth and PHBV production throughout the five repeated cultures of the semi-continuous process were consistent (FIG. 15). The bioreactor was harvested approximately every 24 h, after reaching an average PHBV concentration of 0.94 ±0.20 g/L (as determined by GC-MS). The accumulation of PHBV was seen to lag behind biomass growth, increasing as carbon and nitrogen became limiting (see FIG. 15).

The yields of PHBV extracted from the harvests via hypo-osmotic shock (0.49±0.14 g/L) were significantly lower than the PHBV concentrations in the culture broth determined by GC-MS (0.94±0.20 g/L), indicating significant losses of PHBV during the extraction process. This is likely due to PHBV coming loose from the pellet following centrifugation and being lost when the supernatant is removed.

The depleted nitrogen concentration at each harvest was consistent at 0.17±0.01 g/L. Notably, this is equal to the nitrogen concentration of the diluted hydrolysate medium prior to yeast extract addition. This may indicate that the nitrogen present in the hydrolysate is mostly in a form not easily accessible to H. mediterranei (for example, undigestible proteins).

Adding a Lipid Extraction Step into the Inventive Seaweed-Based Process

Experiments were conducted to investigate the effect of integrating an upstream lipid extraction step into the inventive seaweed-based production process. More specifically, experiments were designed to:
Test the effect of the addition of an upstream lipid extraction process on our biorefinery co-products yields-g of (1) dry agar, (2) glucose in hydrolysate and (3) protein in left over biomass, per 100 g of dry seaweed biomass;
Determine the composition of the lipids extracted from the seaweed;
Assess the efficiency of our lipid extraction process, in % of total lipids that were successfully extracted.
Compare Haloferax mediterranei growth curves and PHA productivities when incubated in seaweed-based growth media prepared after lipid extraction vs seaweed-based growth media with no upstream lipid extraction.

### Methods

Lipid Extraction. 200 grams of dried red seaweed (Gracilaria sp. purchased from Indonesia) were milled using a food processor (Robot Coupe Blixer® 7; particle size of <2 mm), and then split it into 4 sub-samples (50 g each); with 2 of them submitted to lipid extraction. The latter was conducted by combining each of the 50 g samples with 2075 mL of a chloroform:methanol:water solution, comprising 830 mL chloroform, 830 mL methanol and 415 ml water. These mixtures were shaken on a rotary shaker for 30 min at room temperature and then centrifuged (Eppendorf 5910R; 3000 rpm, 10 min, 4° C.). The chloroform and methanolic phases were removed separately and the biomass washed with 1 L of deionized water and centrifuged again at the same conditions aforementioned to remove the solvent. The chloroform phase was stored at −80° C. for lipids and fatty acids determination, while the pelleted biomass phase was oven dried at 60° C. overnight to be used in the next biorefinery step (agar extraction).

2 mL of the chloroform phase containing the lipids were dried under nitrogen gas and the total lipid amount was quantified gravimetrically. The dried aliquot of lipids was methylated to fatty acid methyl esters (FAMEs) by suspending it in BF₃ (7% in methanol) and toluene. The mixture was heated to 100° C. for 45 min and after it cooled down, water was added and the FAMEs were extracted with hexane. The FAMEs were evaluated by gas chromatography coupled with mass spectrometry (QP2010; Shimadzu) with a ZB-Wax capillary column (30 m length, 0.25 mm diameter and 0.25 μm thickness). 1 μL sample was injected at 220° C. using a split of 10 and helium gas at a flow rate of 1 ml min⁻¹. The temperature increased from 60° C. to 240° C. at a rate of 5° C. per minute, and it was kept at 240° C. for 10 min. Supelco 37 Component FAME Mix (Merck) was used as standard and the FAMEs were identified and quantified, respectively, according to retention times and line equation of each FAME of Supleco 37.

Agar extraction. The four 50 g biomass samples (2 with upstream lipid extraction, and 2 with no lipid extraction) were soaked in 1 L of seawater locally sourced in our lab in Western Australia (31.8521° S, 115.7518° E). This seawater was obtained via the ocean pumps and filtration systems of our aquaria facility (Indian Ocean Marine Research Centre Watermans Bay, Western Australia). The seaweed-water mixtures were heated to 95° C. in a water bath (ZZKD), held at this temperature for 3 h, and then centrifuged (Eppendorf 5910R; 4500 rpm, 15 min) for separation of the liquids containing agar, from the solids used for hydrolysate production. The solid fractions were washed again with 1 L of hot seawater (70° C.) and centrifuged again (4500 rpm, 15 min). The washed solids were set aside (for posterior enzymatic treatment and hydrolysate production), while the liquid fractions were combined with the first liquid fractions (containing the agar extracts) and then allowed to gel at room temperature overnight. The gels were frozen at −20° C., thawed at room temperature and then dried in an oven at 60° C. for 48 h. The dried agar was washed with 1 L cold (4° C.) freshwater on a magnetic stirrer for 1 h, filtered through muslin cloth, and then washed again under the same conditions (1 L 4° C. water) and dried at 60° C. for a further 24 h. To remove remaining water, the agar was dehydrated by soaking in acetone (50 mL) for 1 h and drying at 60° C. for a further ˜2.5 h. Agar yields were quantified gravimetrically as g of dried agar per 100 g of dried seaweed biomass.

Acid pre-treatment. Following agar extraction, a pre-treatment process was applied to the solids to make cellulose more accessible to enzymatic digestion. The 4 samples were combined with 500 mL of seawater containing citric acid (25 mM). These seaweed-water mixtures were heated to 120° C. in an autoclave (Biobase BKQ-B75I) for 30 min, cooled to ˜50° C. in a water bath and separated by centrifugation (Eppendorf 5910R; 4500 rpm, 20 min). The solids were stored until further use, while the liquid fractions were submitted to a detoxication process prior to re-mixing with the solids for the enzymatic treatment (see details below).

Detoxification pre-treatment. Since the relatively mild acid pre-treatment (120° C., 25 mM citric acid) generate very low levels of glucose (<0.1 g/L; data not shown), it is possible to perform a detoxication process prior to enzymatic treatment to remove fermentation inhibitors such as phenolic compounds and 5-HMF without the risk of glucose loss. As such, the inventors describe a detoxication process with two treatments (see details below). The first, called over-liming treatment afterwards, involved adjusting the liquid fractions pH from ˜3.5 to 11 using a slurry of calcium hydroxide (All Chemical, Australia) and water (1:1 w/v), agitating the solutions for 30 min on a rotary shaker (Thermo Scientific MaxQ 8000 shaker incubator; 40° C., 150 rpm) and then precipitating the solids via centrifugation (Eppendorf 5910R; 4500 rpm, 20 min). The second, called charcoal treatment afterwards, consisted of adjusting the post over-liming liquids to pH 5 with 2 M HCl and then combining with 2.5% w/v of activated charcoal powder (All Chemical, Australia), agitating the mixtures for 30 min as previously, and centrifuging (as before) to remove the charcoal.

Enzymatic hydrolysis. The pre-treated supernatants were recombined with their respective solids (as obtained after acid pre-treatment) and then exposed to an enzymatic hydrolysis process aimed at breaking down cellulose into glucose. Two cellulolytic enzyme preparations-Celluclast and Viscozyme-were added (0.1 mL each per gram of dry seaweed biomass), and the mixtures incubated at 50° C. and 130 rpm in a water bath for 20 h. Finally, the mixtures were separated by centrifugation (4500 rpm, 20 min) to separate the spent seaweed biomass (solids) from the hydrolysates (liquids). The latter had glucose concentrations (in g/L) measured using D-glucose HK assay kits (Megazyme®). The solids were washed with distilled water (1 L), dried in an oven (60° C.) overnight, and then handed to Agrifood Technology (Perth, Australia) for determination of total protein, lipids and carbohydrates contents. These same analyses were also conducted in the raw seaweed biomass (Gracilaria sp.) for comparison purposes.

Fermentation media preparation. The four liquid hydrolysates had their pH adjusted to 7 with 2 M NaOH and combined with the following amounts of salts per L (adapted from ATCC Medium 1176 formulation to reach a final salinity of 170 ppt): NaCl, 124 g; MgCl2·6H2O, 16 g; CaCl2. 2H2O, 0.8 g; KCl, 3 g; NaHCO₃, 0.16 g; NaBr, 0.4 g. Trace metals were supplied with the addition of 1 mL per litre of SL-6 trace metals mix (FIG. 3). This medium was then chilled overnight to 4° C. and centrifuged in batches (8500 rpm, 15 min, 4° C.) to remove any remaining precipitate. The four growth media were then diluted (50% strength) with a solution containing salts as provided for in ATCC Medium 1176 (FIG. 2; prepared without glucose or yeast extract) containing 1 mL per litre of SL-6 metals mix. Finally, yeast extract at 2 g/L concentration were added to all four growth media.

Fermentation-shake flask experiment. To test whether the addition of our lipid extraction step had any influence in the fermentability and PHA production of our process, we performed a shake flask experiment. A total of 12 250 mL shake flask cultures were prepared, 6 with 60 mL of seaweed-derived media exposed to lipid extraction, and 6 with 60 mL of seaweed-derived media not exposed to lipid extraction.

The inoculum for this fermentation experiment was prepared by growing a single colony of Haloferax mediterranei (ATCC 33500) in a 50 mL Falcon tube containing 5 mL of a solution containing salts as provided for in ATCC Medium 1176 at 40° C. and 150 rpm for 24 h (Thermo Scientific MaxQ 8000). After that, this 5 mL culture was transferred into 250 mL flask containing 45 mL of the same medium and cultured for a further 72 h, under the same fermentation conditions. The 50 mL culture was centrifuged (Eppendorf 5910R; 4500 rpm, 20 min, 4° C.) and the cells were resuspended in the salt solution described above. The cell suspension was used to inoculate the 12 seaweed growth media cultures to a starting optical density (600 nm wavelength (OD₆₀₀); measured with Eppendorf Kinetic® BioSpectrometer) of ˜0.8.

The cultures were incubated at the same conditions as the inoculum (40° C., 150 rpm), with growth assessed every 12-24 h by optical density OD₆₀₀ measurements. Cells were harvested by centrifugation (Eppendorf 5910R; 10100 rpm, 20 min, 4° C.) after growth had ceased or all glucose had been consumed (assessed using an Abbott FreeStyle Optium Neo glucose monitor) with final polyhydroxyalkanoate (PHA) concentrations determined by both GC-MS and direct gravimetric measurements.

GC-MS measurement of the final PHBV concentrations (in g/L) were made with 7 mL samples of the fermentation broth. These were centrifuged (Eppendorf 5910R; 10100 rpm, 20 min, 4° C.), and the cell pellets lyophilized. Freeze-dried cell pellets were weighed and then subjected to methanolysis with 2 mL of 15% sulfuric acid in methanol and 2 mL of chloroform at 100° C. for 2 h 20 min. The chloroform phase containing hydroxyalkanoate methyl esters was then removed for analysis via GC-MS, using benzoic acid as an internal standard. The butyric and valeric methyl esters were evaluated by gas chromatography coupled with mass spectrometry (QP2010; Shimadzu) with a ZB-Wax capillary column (30 m length, 0.25 mm diameter and 0.25 μm thickness). 1 μL sample was injected at 250° C. using a split of 20 and helium as a gas carrier. The temperature increased from 60° C. to 250° C. in a rate of 20° C. per minute, and it was kept at 250° C. for 5 min. Quantification was performed by comparison to a standard curve prepared with PHBV of known purity and composition.

Direct measurement of the final PHBV concentration (in g/L) were also made by extracting PHAs from 40 mL volumes from each culture. These were centrifuged (Eppendorf 5910R; 10100 rpm, 20 min, 4° C.) and the supernatant discarded. The cell pellets were frozen at −20° C. overnight. Pellets were then thawed and combined with 40 mL reverse osmosis deionized water. containing 0.1% (w/v) sodium dodecyl sulphate (SDS). The cell pellets were resuspended and agitated at 150 rpm at 40° C. for 1 h. The suspensions were centrifuged again, the supernatant discarded, and the pellets washed in a further 40 mL of 0.1% SDS. The centrifugation and washing process was then repeated twice using reverse osmosis deionized water without SDS. The pellets were then transferred to pre-weighed trays and dried for 24 h at 60° C. in an oven before weighing to determine dry weight.

### Results and Discussion

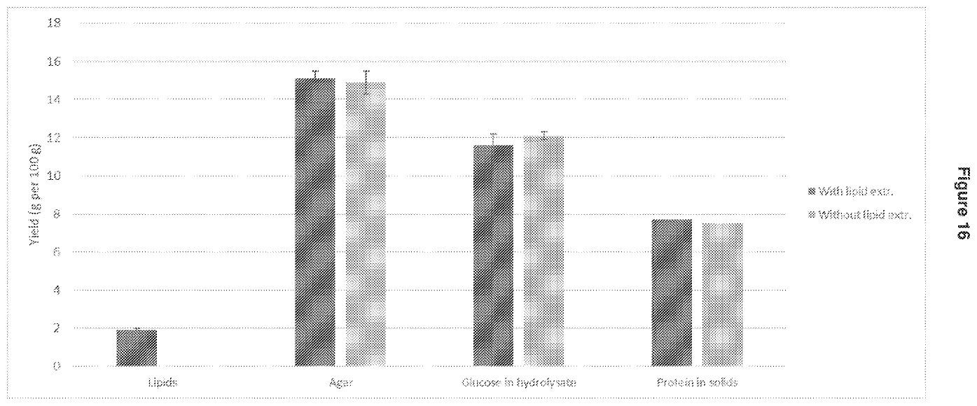

Biorefinery. The “biorefinery” process outlined above, including hydrocolloid, protein feed and PHA production, was successfully performed both with and without lipid extraction. The addition of the upstream lipid extraction step had no significant effect on the final yields of our biorefinery products (FIG. 16). It appears that this solvent extraction process did not leave any impurities affecting the efficiency of our hydrolysis process, nor changed the biochemistry of the seaweed by removing and/or degrading the seaweed's hydrocolloid (agar) and cellulose (which is broken down into glucose during enzymatic hydrolysis).

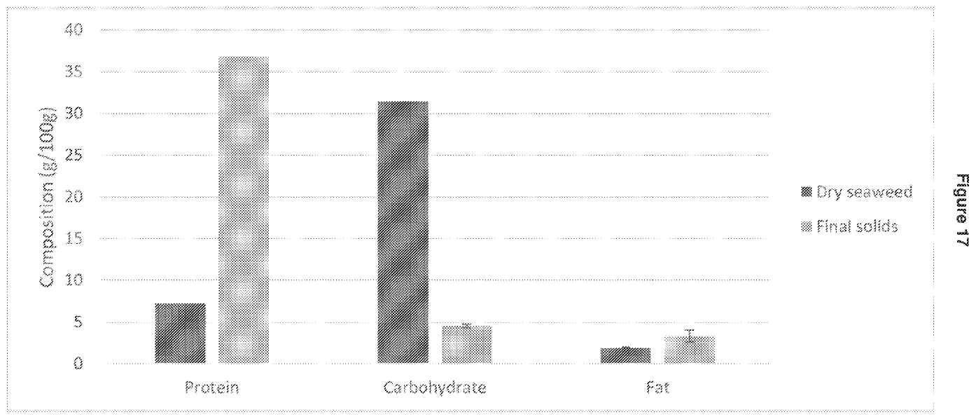

Co-products obtained from our complete seaweed biorefinery process included a lipid extract (yield=1.8±0.3 g of lipid per 100 g of dry seaweed biomass), agar (15.3±0.4 g of dry agar per 100 g of dry seaweed biomass), a glucose-rich hydrolysate (11.6±0.4 g of glucose per 100 g of dry seaweed biomass), and a protein-rich solid (7.7 g of protein per 100 g of dry seaweed biomass). The latter contained a higher protein content (36.8 g of protein per 100 g on dry-weight basis) than the raw seaweed biomass (7.2 g of protein per 100 g on dry-weight basis), making it an attractive food/feed source, particularly suitable in aquaculture (due to the presence of salt). This boost in protein content is due to the removal of ˜97% of the carbohydrates from the raw seaweed biomass (30.6 of carbohydrates per 100 g of dry seaweed biomass) via agar removal and cellulose hydrolysis (FIG. 17).

In terms of the fatty acid composition of the extracted lipids, nearly 99% was saturated fatty acids (palmitic, stearic and myristic acids), with only one unsaturated fatty acid (oleic acid) detected (Table 11). Given the low yield and market value of the extracted lipids from this particular seaweed, it may be more beneficial to omit the lipid extraction step so that the lipids remain in the final solids and boost its nutritional value as a potential aquaculture feed. It is also worth noting that this lipid extract process has scope for improvement with 63.5±9.4% of the seaweed lipid content being successfully extracted. This could be improved by repeating the solvent extraction one or more times.

TABLE 11
Fatty acids composition of lipid extracts (n = 2)
from the red seaweed Gracilaria sp.

| Type | Name | % of total |
|---|---|---|
| Saturated | Palmitic acid (hexadecanoic acid; | 91.6 ± 13.5 |
| | C16:0) | |
| | Stearic acid (octadecanoic acid; | 4.9 ± 0.7 |
| | C18:0) | |
| | Myristic acid (tetradecanoic acid; | 2.2 ± 0.5 |
| | C14:0) | |
| Monounsaturated | Oleic acid (9-octadecenoic acid; | 1.3 ± 0.5 |
| | C18:1Δ9) | |

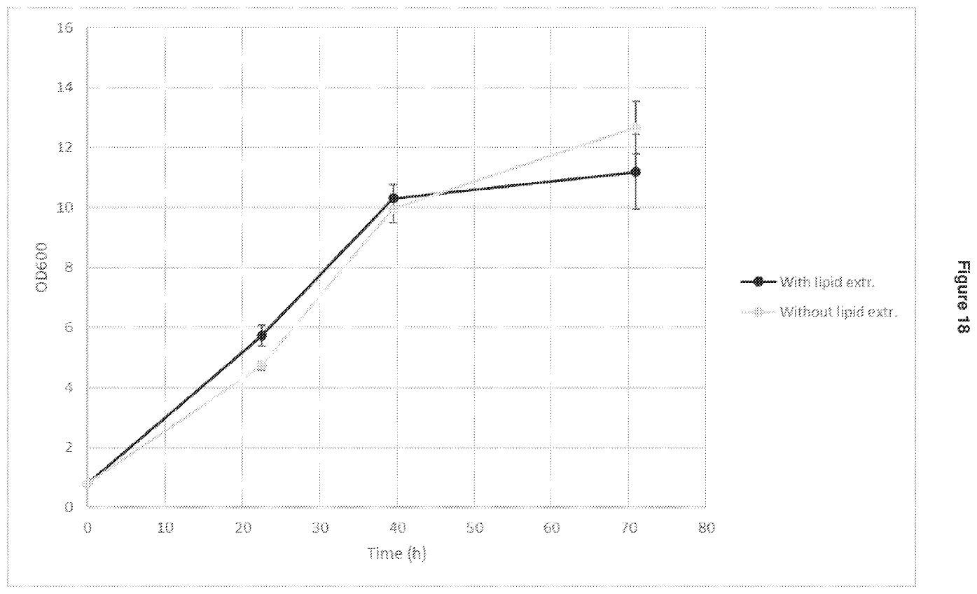

Fermentation-cell growth and PHA production. Cell growth patterns (FIG. 18) were similar for both kinds of fermentation media used in the study: (1) made with the seaweed hydrolysates produced after lipid extraction (50% strength, 2 g/L yeast extract), and (2) made with seaweed hydrolysate produced without any preceding lipid extraction (50% strength, 2 g/L yeast extract). Notably, the cultures in this experiment grew significantly faster and reached higher cell densities than those described in the previous investigation (Testing the inventive Seawater-based Process at Pilot Scale) (see FIG. 13). While some of this difference may be due to different glucose concentrations in the media (7.2±0.8 g/L vs 5.9±0.5 g/L in 50% strength in previous experiment), it is most likely attributed to using a more active growth inoculum and inoculating to a higher initial cell density (OD₆₀₀ 0.8 vs 0.5).

The resulting PHA productivity were also similar for both kinds of growth media (i.e. with and without lipid extraction; Table 12). It is worth noting that the concentration of PHBV is lower when analysed by GC-MS compared to mass of extracted PHBV. This is due to impurity of the extracted material, with the average (±std) purity of the extracted PHBV being equal to ˜76±5%. The potential impurities include non-lysed cells and cell debris such as protein and membrane lipids.

Overall, the average yield of PHBV per 100 g of dry seaweed (based on extracted mass) was 3.8±0.3 g. Potential strategies to improve this metric include:
(i) Investigation of other seaweed varieties which may produce higher yields of fermentable sugars;
(ii) Including a protease digestion step during hydrolysate production to improve carbon and nitrogen concentration;
(iii) Better removal of fermentation inhibitors.

TABLE 12
Resulting PHBV yields (in grams by L of medium) and cell %
of PHBV as estimated by direct PHA extractions and GC-MS.

| | By extracted | | |
|---|---|---|---|
| | PHA mass | By GC-MS analysis | |
| | PHBV | PHBV | |
| | concentration | concentration | Cell % |
| Treatment | (g/L) | (g/L) | PHBV |
| With lipid extraction | 2.15 ± 0.16 | 1.64 ± 0.08 | 26.1 ± 0.9 |
| Without lipid extraction | 2.36 ± 0.16 | 1.83 ± 0.06 | 27.3 ± 0.7 |

Seawater Lysis of Haloferax mediterranei for PHBV Extraction

The inventors PHA extraction process is based on the lysis of halophilic cells (˜170 ppt) via hypo-osmotic shock when exposing such salt-containing cells to a solution of lower salt content.

In previous embodiments, H. mediterranei cells were lysed via hypo-osmotic shock by exposing them to solutions comprising reverse osmosis (RO) water. Nonetheless, the use of freshwater (0 ppt) at scale can be expensive and unsustainable, making seawater (35 ppt) an attractive alternative.

Investigation was conducted to assess the efficiency of solutions comprising different ratios of RO water and seawater (salinity range=0-35 ppt) to lyse H. mediterranei cells (˜170 ppt) and effectively extract the intracellular PHAs.

### Methods

Fresh H. mediterranei cells were harvested from 9×40 mL fermentation broth samples (poured inside 50 mL Falcon tubes) via centrifugation (15000 g for 10 min) and discard of supernatant.

The resulting 9 cell pellets were resuspended in 40 ml of solutions containing 0.1% SDS and different levels of seawater: 0% (100% RO water; 0 ppt), 12.5%, 25%, 37.5%, 50%, 62.5%, 75%, 87.5%, and 100% (pure seawater; 35 ppt). These solutions were made by mixing RO water with filtered seawater locally sourced at Indian Ocean Marine Research Centre, Watermans Bay, Western Australia. The suspensions were then agitated (250 rpm) for 1 h at 25° C. inside an incubator, centrifuged (15000 g for 10 min) and the supernatant discarded. The resulting pellets had their colour (as a proxy for cell lysis level) inspected and documented via a photograph. Since whole H. mediterranei cells are pink and PHA granules are white, less pink pellets indicate better cell lysis.

### Results & Discussion

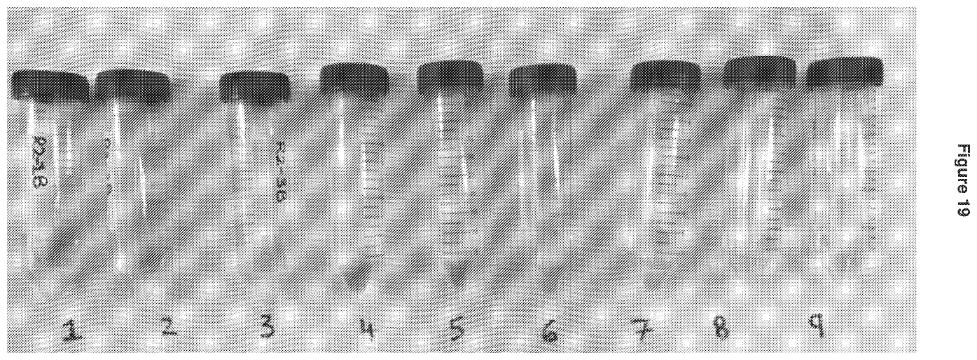

Different degrees of cell lysis were observed, with better lysis levels occurring when submerging the halophilic cells in seawater rather than freshwater. This was evident by observing the colour of the resulting pellets (FIG. 19) which had the strongest pink colour in the freshwater treatment, the whitest appearance in the seawater treatment, and a pink colour fading away from solutions with higher freshwater proportions to solutions with higher levels of seawater.

Our hypothesis is that the better lysis in seawater is due to the salt ions preventing free DNA strands and proteins from interacting and binding together, leading to a better exposure of the salt-containing cells (˜170 ppt) to the solution (<35 ppt). The higher viscosity under lower salinity solutions were clearly visible, with many cells entrapped in some highly viscous portions of the media.

### OTHER

Reference to cited material or information contained in the text should not be understood as a concession that the material or information was part of the common general knowledge or was known in Australia or any other country.

Each document, reference, patent application or patent cited in this text is expressly incorporated herein in their entirety by reference, which means that it should be read and considered by the reader as part of this text. That the document, reference, patent application, or patent cited in this text is not repeated in this text is merely for reasons for conciseness.

Optional embodiments of the present invention may also be said to broadly consist in the parts, elements and features referred to or indicated herein, individually or collectively, in any or all combinations of two or more of the parts, elements or features, and wherein specific integers are mentioned herein which have known equivalents in the art to which the invention relates, such known equivalents are deemed to be incorporated herein as if individually set forth.

It is to be appreciated that reference to “one example” or “an example” of the invention is not made in an exclusive sense. Accordingly, one example may exemplify certain aspects of the invention, whilst other aspects are exemplified in a different example. These examples are intended to assist the skilled person in performing the invention and are not intended to limit the overall scope of the invention in any way unless the context clearly indicates otherwise.

It is to be understood that the terminology employed above is for the purpose of description and should not be regarded as limiting. The described embodiment is intended to be illustrative of the invention, without limiting the scope thereof. The invention is capable of being practised with various modifications and additions as will readily occur to those skilled in the art.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

Various substantially and specifically practical and useful exemplary embodiments of the claimed subject matter are described herein, textually and/or graphically, including the best mode, if any, known to the inventors for carrying out the claimed subject matter.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in the specification, individually or collectively and any and all combinations or any two or more of the steps or features.

The inventor(s) expects skilled artisans to employ such variations as appropriate, and the inventor(s) intends for the claimed subject matter to be practiced other than as specifically described herein. Accordingly, as permitted by law, the claimed subject matter includes and covers all equivalents of the claimed subject matter and all improvements to the claimed subject matter. Moreover, every combination of the above described elements, activities, and all possible variations thereof are encompassed by the claimed subject matter unless otherwise clearly indicated herein, clearly and specifically disclaimed, or otherwise clearly contradicted by context.

The present invention is not to be limited in scope by the specific embodiments described herein, which are intended for the purpose of exemplification only. Functionally equivalent products, compositions and methods are clearly within the scope of the invention as described herein.

The use of any and all examples, or exemplary language (e.g., “such as” or “for example”) provided herein, is intended merely to better illuminate one or more embodiments and does not pose a limitation on the scope of any claimed subject matter unless otherwise stated. No language in the specification should be construed as indicating any non-claimed subject matter as essential to the practice of the claimed subject matter.

The use of words that indicate orientation or direction of travel is not to be considered limiting. Thus, words such as “front”, “back”, “rear”, “side”, “up”, down”, “upper”, “lower”, “top”, “bottom”, “forwards”, “backwards”, “towards”, “distal”, “proximal”, “in”, “out” and synonyms, antonyms and derivatives thereof have been selected for convenience only, unless the context indicates otherwise. The inventor(s) envisage that various exemplary embodiments of the claimed subject matter can be supplied in any particular orientation and the claimed subject matter is intended to include such orientations.

The use of the terms “a”, “an”, “said”, “the”, and/or similar referents in the context of describing various embodiments (especially in the context of the claimed subject matter) are to be construed to cover both the singular and the plural, unless otherwise indicated herein or clearly contradicted by context. The terms “including,” “having,” “including,” and “containing” are to be construed as open-ended terms (i.e., meaning “including, but not limited to,”) unless otherwise noted.

Throughout the specification and claims, unless the context requires otherwise, the word “comprise” or variations such as “comprises” or “comprising”, will be understood to imply the inclusion of a stated integer or group of integers but not the exclusion of any other integer or group of integers.

Moreover, when any number or range is described herein, unless clearly stated otherwise, that number or range is approximate. Recitation of ranges of values herein are merely intended to serve as a shorthand method of referring individually to each separate value falling within the range, unless otherwise indicated herein, and each separate value and each separate sub-range defined by such separate values is incorporated into the specification as if it were individually recited herein. For example, if a range of 1 to 10 is described, that range includes all values there between, such as for example, 1.1, 2.5, 3.335, 5, 6.179, 8.9999, etc., and includes all sub-ranges there between, such as for example, 1 to 3.65, 2.8 to 8.14, 1.93 to 9, etc.

Accordingly, every portion (for example, title, field, background, summary, description, abstract, drawing figure, etc.) of this application, other than the claims themselves, is to be regarded as illustrative in nature, and not as restrictive; and the scope of subject matter protected by any patent that issues based on this application is defined only by the claims of that patent.

In the figures, incorporated to illustrate features of a non-limiting example embodiment, like reference numerals are used to identify like parts throughout the figures.

1.-45. (canceled)

46. A process for producing polyhydroxyalkanoates from macroalgae, comprising the steps of:
forming a macroalgal mixture from macroalgae and saltwater;
enzymatically hydrolysing the macroalgae mixture to form a macroalgal hydrolysate;
producing a growth medium comprising the macroalgal hydrolysate;
fermenting the growth medium using halophilic microbes capable of producing polyhydroxyalkanoates in a non-sterile fermentation process; and
extracting the polyhydroxyalkanoates from within the halophilic cells using a saltwater-based lysis process.

47. A process according to claim 46, wherein the saltwater comprises seawater and/or water with added salts.

48. A process according to claim 46, wherein the macroalgae comprises cultivated Gracilariaceae, Solieriaceae, Bangiaceae, Ulvaceae, Alariaceae, Lessoniaceae or Laminariaceae.

49. A process according to claim 48, wherein the macroalgae comprises a species of macroalgae selected from the group comprising: Gracilaria spp., Gracilariopsis spp., Kappaphycus spp., Eucheuma spp., Betaphycus spp., Porphyra spp., Pyropia spp., Gelidium spp., Asparagopsis spp., Ulva spp., Undaria spp., Saccharina spp., or Ecklonia spp.

50. A process according to claim 46, wherein the halophilic microbes comprise microbes that grow in salt concentrations of at least 150 g/L.

51. A process according to claim 46, wherein the halophilic microbe is a Haloferax spp. or Haloferax mediterranei.

52. A process according to claim 46, wherein the macroalgal mixture is formed from wet macroalgae and saltwater,
wherein the macroalgae is broken down into smaller portions prior to forming the macroalgal mixture, and
wherein the smaller portions of macroalgae comprise particles of less than approximately 2 mm in diameter.

53. A process according to claim 52, wherein hydrocolloids are extracted and removed from the macroalgal mixture.

54. A process according to claim 53, wherein the macroalgal mixture is treated with a weak acid, and
wherein the weak acid comprises citric acid.

55. A process according to claim 46, wherein the macroalgal mixture is enzymatically hydrolysed to form the macroalgal hydrolysate which comprises an aqueous phase and a solid phase, and wherein the solid phase is removed from the macroalgal hydrolysate, and
wherein the macroalgal hydrolysate is detoxicated.

56. A process according to claim 46, wherein the step of fermenting the growth medium using halophilic microbes, in a non-sterile fermentation process, comprises inoculating the growth medium with halophilic microbes capable of producing polyhydroxyalkanoates to form a fermentation culture, without sterilisation of the growth medium or the fermentation system.

57. A process according to claim 56, wherein the fermentation culture is grown in a continuous or semi-continuous fermentation system.

58. A process according to claim 57, wherein the continuous fermentation system comprises two or more fermentation reactors operating sequentially or in a series or cascade, and
wherein additional growth media is added to the fermentation culture during fermentation.

59. A process according to claim 58, wherein the carbon-nitrogen ratio of the fermentation culture inside the reactors increases from the first fermentation reactor to a last fermentation reactor.

60. A process according to claim 58, wherein the carbon-nitrogen ratio of the fermentation culture inside each reactor is kept at constant ratio via feeding of additional growth media into each reactor,
wherein, the continuous fermentation system comprises inlet feeds providing growth media to the fermentation reactors and outlet feeds, pumping fermentation culture from the first reactor, through each subsequent reactor, to a last reactor, and
wherein an outlet feed from the last reactor is set at a flow rate equal to the sum of all inlet feed flow rates in the continuous fermentation system.

61. A process according to claim 56, wherein acid and base are added during fermentation to maintain the fermentation culture at approximately pH 7.0, and at a temperature of approximately 40° C.

62. A process according to claim 46, wherein the step of extracting the polyhydroxyalkanoates from within the halophilic cells using a saltwater-based lysis process, comprises harvesting the cell biomass by separating it from the fermentation culture by centrifugation or filtration.

63. A process according to claim 62, wherein the separated halophilic cells are submersed in a saltwater-based solution to lyse them, and
wherein the saltwater-based solution comprises seawater or water with added salts.

64. A process according to claim 63, wherein the saltwater-based solution comprises additional chemical or enzymatic agents, and
wherein the additional chemical or enzymatic agents comprises sodium dodecyl sulfate (SDS).

65. A process according to claim 62, wherein polyhydroxyalkanoates are recovered from the halophilic cells by submersing them in, followed by centrifuging or filtering them from, the saltwater-based solution one or more times and removing the aqueous phase.
